(19) United States
(12) Patent Application Publication
(10) Pub. No.: US 2026/0240602 A1
(43) Pub. Date: Aug. 20, 2026

(54) MAGNETIC LOCATION DETECTION

(71) Applicant: University of Pittsburgh - Of the Commonwealth System of Higher Education, Pittsburgh, PA (US)

(72) Inventors: William W. Clark, Wexford, PA (US); Yifan Zhang, Pittsburgh, PA (US); Youngjae Chun, Wexford, PA (US); Bryan W. Tillman, Delaware, OH (US); Sung Kwon Cho, Pittsburgh, PA (US)

(73) Assignee: University of Pittsburgh - Of the Commonwealth System of Higher Education, Pittsburgh, PA (US)

(21) Appl. No.: 18/859,506

(22) PCT Filed: May 10, 2023
(86) PCT No.: PCT/US2023/021757

§ 371 Date: Oct. 23, 2024

Related U.S. Application Data
(60) Provisional application No. 63/340,309, filed on May 10, 2022.

(51) Int. Cl. A61B 34/20; A61F 2/90; A61F 2/962

(52) U.S. Cl. CPC A61B 34/20; A61F 2/90; A61F 2/962; A61B 2034/2051

(57) ABSTRACT

This disclosure is directed to systems for magnetic location detection, and methods of using the magnetic location detection systems disclosed herein, particularly as applied to the deployment of medical devices in the body of a patient. In some examples, the location direction system comprises a reference magnet and a detector. In some examples, the location detection system can be utilized along with a perfusion device, such as an internally implantable stent. In some examples, the system can be utilized to guide the perfusion device to a desired internal implantation site, and in some examples, can include correction factors to adjust for magnetic interference from hard and soft magnetic sources.

### CROSS REFERENCE TO RELATED APPLICATION

This application claims the benefit of U.S. Provisional Patent Application No. 63/340,309, filed on May 10, 2022, which is incorporated by reference herein in its entirety.

### ACKNOWLEDGMENT OF GOVERNMENT SUPPORT

This invention was made with government support under Grant No. EB022591 awarded by The National Institutes of Health and with government support under Grant No. W81XWH-16-2-0062 awarded by the United States Army Medical Research and Materiel Command. The government has certain rights in the invention.

### FIELD

The present disclosure relates to magnetic location detection devices and methods for using magnetic location detection devices, such as may be used to position a medical device within the body of a patient.

### BACKGROUND

The human vasculature can suffer from various injuries, such as arterial or venous punctures or tears, which in turn cause internal hemorrhage. Treating these injuries requires prompt establishment of hemostasis to control hemorrhage. In cases where the vascular injury occurs deep within the patient's body, hemostasis may be difficult to achieve with compression treatment or a bandage. Instead, a stent may be deployed internally to cover and seal the injury. In this procedure, the stent may be advanced through the vasculature of the patient to the site of the injury and deployed to cover the injury. To facilitate proper positioning of the stent during the treatment procedure, methods of detecting stent location within the body of the patient are required.

### SUMMARY

Disclosed herein are perfusion devices including a deployable stent for the treatment of vascular injuries, as well as magnetic location detection systems for positioning the same within the body of a patient during a treatment procedure. The disclosed magnetic location detection systems can include a reference magnet, or in specific examples, an electromagnet, that generates a known magnetic field. The perfusion devices can be deployed along with a detector, such as a magnetometer, that measures the strength and vector of the known magnetic field to determine the position of the detector relative to the reference magnet. When the reference magnet has a known position relative to the body of the patient, and the detector has a known position relative to the deployable stent, the position of the stent relative to the patient's anatomy can be determined. Also disclosed herein are various methodologies for correcting for measurement error and interference to improve accuracy of stent deployment.

Certain examples concern a medical assembly, comprising a delivery apparatus comprising a sheath and a guidewire, configured to be advanced through vasculature within a body of a patient. The medical assembly also includes a perfusion device having a radially expandable stent comprising a frame and a sealing cover disposed along the frame, a magnet, and a detector comprising a magnetometer. The magnet is configured to be positioned external to the body of the patient and to generate a known magnetic field. The stent and the detector are positioned on the guidewire and configured to be advanced along the guidewire and deployed from the sheath. The magnetometer is configured to measure the known magnetic field and to identify a position of the detector relative to the magnet.

Certain examples concern a method for treating a vascular injury of a patient, comprising placing a magnet at a reference point on a body of the patient and generating a reference magnetic field and an origin point for the reference magnetic field. The method also comprises determining a location of the vascular injury relative to the origin point of the reference magnetic field and inserting a medical assembly comprising a perfusion stent and a detector into the patient's vasculature and advancing the medical assembly towards the location of the vascular injury. The method also comprises using the detector to measure the reference magnetic field and determine a position of the medical assembly relative to the origin point of the reference magnetic field and deploying the perfusion stent once the medical assembly has reached the location of the vascular injury.

Certain examples concern a magnetic location detection system, comprising a reference magnet, a detector including a magnetometer, and a microcontroller in communication with the detector. The reference magnet is placed external to a patient and configured to generate a reference magnetic field with a known origin, a known magnitude, and a known direction. The magnetometer is configured to measure a first magnitude and a first direction of the reference magnetic field at a first position. The microcontroller is configured to determine the first position of the magnetometer relative to an origin of the reference magnetic field using a measurement of the first magnitude and the first direction of the reference magnetic field.

The foregoing and other objects, features, and advantages of the invention will become more apparent from the following detailed description, which proceeds with reference to the accompanying figures.

### BRIEF DESCRIPTION OF THE DRAWINGS

FIG. 1 is a schematic diagram of an aortic perfusion device according to one example, deployed in the vasculature of a patient.

FIG. 2 is an illustration of the aortic perfusion device of FIG. 1, showing the delivery catheter and the stent in a radially expanded configuration.

FIG. 3 is an illustration of a fluid-inflatable perfusion device according to one example.

FIG. 4 is an illustration of the fluid-inflatable perfusion device of FIG. 3 with added electromagnetic sensors.

FIG. 5 is a schematic illustration of the fluid-inflatable perfusion device of FIG. 4, shown relative to a reference portion of a patient's anatomy.

FIG. 6A is a schematic diagram of a blood pressure sensor according to one example.

FIG. 6B is a schematic diagram of an electrode portion of the blood pressure sensor of FIG. 6A.

FIG. 7A is a schematic diagram showing the assembly of a blood pressure sensor according to another example.

FIG. 7B is a schematic diagram showing the blood pressure sensor of FIG. 7B in the assembled state.

FIG. 8 is a diagram of an RLC circuit according to one example.

FIG. 9 is a plot of the frequency response of the RLC circuit example shown in FIG. 8.

FIG. 10 is a perfusion device including a radially expandable stent according to one example.

FIG. 11 is a top view of the stent of the perfusion device of FIG. 10.

FIG. 12A is a schematic illustration of an injury to the aorta of a patient.

FIG. 12B shows the deployment of the perfusion device of FIG. 10 to seal the injury shown in FIG. 12A.

FIG. 12C shows the retraction of the perfusion device of FIG. 10 into a delivery apparatus following the treatment of the injury of FIG. 12A.

FIG. 13 is an illustration of a perfusion device according to another example, having a stent and an inflatable balloon to radially expand the stent.

FIG. 14A is an illustration of the radially expandable frame of a perfusion device according to another example.

FIG. 14B is an illustration of a perfusion device according to the example of FIG. 14A, showing the radially expandable frame and a sleeve mounted to the frame.

FIG. 14C is an end view of the perfusion device of FIG. 14B.

FIG. 14D is an illustration of the perfusion device of FIG. 14B, contained in a delivery sheath in a radially compressed configuration.

FIG. 15 is a plot of signal output at the monitor vs. the lateral distance between an RFID and an antenna.

FIG. 16 a representative illustration of a system for magnetic location detection.

FIG. 17A is an illustration of a perfusion device according to another example.

FIG. 17B is an illustration of the upper vasculature of a patient.

FIG. 17C is a diagram showing the placement of the perfusion device of FIG. 17 to avoid obstruction of perfusion to abdominal organs.

FIG. 18 is a schematic illustration of a reference magnet, a magnetic coordinate system, and a patient with a patient coordinate system aligned with the magnetic coordinate system.

FIG. 19A is an illustration of a detector according to one example, including one or more sensors.

FIG. 19B is an illustration of a perfusion device, including the detector of FIG. 19A.

FIG. 20A is an illustration of the magnetic field for an example cylindrical magnet in a vertical plane.

FIG. 20B is an illustration of the magnetic field for an example cylindrical magnet in three dimensions.

FIG. 21 is a diagram of the rotational components, yaw, pitch, and roll, of a magnet in a three-dimensional coordinate system.

FIG. 22 is a schematic illustration of a magnetic location detection test system according to one example.

FIG. 23 shows a prototype of a magnetic location detection test system according to the example of FIG. 22.

FIG. 24A is a plot of the measured position of a detector in the X direction against the true position of the detector in the X direction.

FIG. 24B is a plot of the measured position of a detector in the Y direction against the true position of the detector in the Y direction.

FIG. 24C is a plot of the measured position of a detector in the Z direction against the true position of the detector in the Z direction.

FIG. 25A shows a prototype magnetic location detection system according to one example, with sources of magnetic interference introduced.

FIG. 25B is a plot showing electromagnetic measurements without interference, with uncorrected interference, and with corrected interference as a function of position.

FIG. 26A is a plot showing correction of the X-axis component of electromagnetic interference as a function of position along the X-axis.

FIG. 26B is a plot showing correction of the Y-axis component of electromagnetic interference as a function of position along the X-axis.

FIG. 26C is a plot showing correction of the Z-axis component of electromagnetic interference as a function of position along the X-axis.

FIG. 27A is an illustration of a test setup to measure the magnetic field impact of example foreign objects.

FIG. 27B is a plot of the results of the magnetic field impact of the foreign objects shown in FIG. 27A.

FIG. 28A shows a test assembly for measuring the impact of yaw, pitch and roll of a magnet in a three-dimensional system.

FIG. 28B shows the test assembly of FIG. 28A with the magnet rotated around the Z-axis.

FIG. 29 is an illustration of a test assembly for measuring the impact of yaw, pitch, and roll on a magnetic location detection system, in the presence of magnetic interference.

FIG. 30A is a plot of the X-axis component test results from the use of the test assembly of FIG. 29.

FIG. 30B is a plot of the Y-axis component test results from the use of the test assembly of FIG. 29.

FIG. 30C is a plot of the Z-axis component test results from the use of the test assembly of FIG. 29.

### DETAILED DESCRIPTION

### General Considerations

For purposes of this description, certain aspects, advantages, and novel features of the examples of this disclosure are described herein. The disclosed methods, apparatuses, and systems should not be construed as limiting in any way. Instead, the present disclosure is directed toward all novel and nonobvious features and aspects of the various disclosed examples, alone and in various combinations and sub-combinations with one another. The methods, apparatuses, and systems are not limited to any specific aspect or feature or combination thereof, nor do the disclosed examples require that any one or more specific advantages be present or problems be solved.

Features, integers, characteristics, compounds, chemical moieties or groups described in conjunction with a particular aspect, example or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing examples. The invention extends to any novel one, or any novel combination, of the features disclosed in this specification (including any accompanying claims, abstract and drawings), or to any novel one, or any novel combination, of the steps of any method or process so disclosed.

Although the operations of some of the disclosed methods are described in a particular, sequential order for convenient presentation, it should be understood that this manner of description encompasses rearrangement, unless a particular ordering is required by specific language. For example, operations described sequentially may in some cases be rearranged or performed concurrently. Moreover, for the sake of simplicity, the attached figures may not show the various ways in which the disclosed methods can be used in conjunction with other methods. As used herein, the terms “a”, “an”, and “at least one” encompass one or more of the specified element. That is, if two of a particular element are present, one of these elements is also present and thus “an” element is present. The terms “a plurality of” and “plural” mean two or more of the specified element.

As used herein, the term “and/or” used between the last two of a list of elements means any one or more of the listed elements. For example, the phrase “A, B, and/or C” means “A”, “B,”, “C”, “A and B”, “A and C”, “B and C”, or “A, B, and C.”

As used herein, the term “coupled” generally means physically coupled or linked and does not exclude the presence of intermediate elements between the coupled items absent specific contrary language.

### Magnetic Location Detection

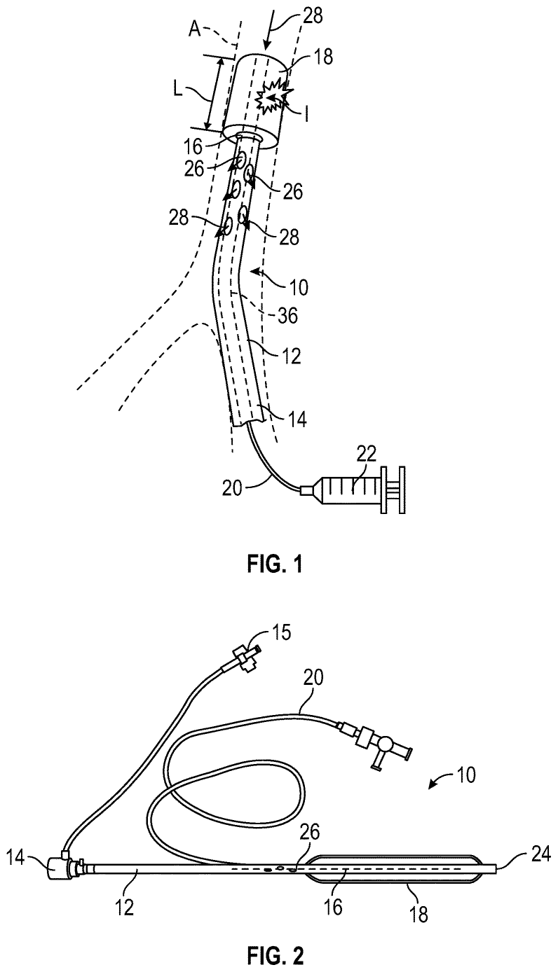

FIG. 1 shows a schematic representation of an implantable perfusion device 10, according to one example, implanted in the descending aorta A. FIG. 2 shows a working example of the perfusion device 10. As shown in FIG. 1, the perfusion device 10 can be implanted adjacent an injury to the aorta (e.g., a ruptured portion of the aorta) to prevent or minimize bleeding from the vessel while still allowing blood to perfuse through the device. Although the perfusion device 10 is described in connection with treating an injury to the aorta, it should be understood that the perfusion device 10 also can be implanted in other blood vessels, as well other tubular organs of the body.

The perfusion device 10 in the illustrated example comprises an elongated shaft 12 having a proximal end portion 14 and a distal end portion 16. An expandable sealing member in the form of an inflatable balloon 18 is mounted on the distal end portion 16 of the shaft 12. An inflation conduit 20 has a distal end fluidicly connected to the balloon 18 and a proximal end fluidicly connected to a source of an inflation fluid, such as the illustrated syringe 22. In use, the syringe 22 transfers a pressurized inflation fluid (e.g., saline) to the balloon to inflate the balloon, as described in greater detail below. The balloon 18 is configured such that when it is inflated, the outer surface of the balloon can contact the inner wall of the aorta A and create a seal around an injury to the vessel to stop or minimize bleeding.

Instead of providing a separate inflation conduit 20, the shaft 12 can be formed with a separate inflation lumen that extends from the balloon 18 to a proximal end of the shaft outside the body. The proximal end of the inflation lumen can be fluidicly connected to a source of an inflation fluid (e.g., a syringe 22) to pump the inflation fluid through the inflation lumen and into the balloon 18.

The shaft 12 has a plurality of perfusion ports 26 (sometimes called apertures 26) proximal to the balloon 18. The shaft 12 has a lumen, or internal passageway, 36 that extends lengthwise of the shaft from a distal opening at a distal end 24 to a location proximal to the ports 26. The perfusion ports 26 are in fluid communication with the lumen of the shaft. Thus, upon deployment within the aorta, a flow path for blood is established through the shaft, in the direction indicated by arrows 28. As shown in FIG. 2, the inflation conduit 20 can enter the lumen 36 at a location intermediate to the port 26 furthest in the proximal direction and the proximal end portion 14 of the shaft 12. Alternatively, the lumen 36 can extend the entire length of the shaft 12. The perfusion device 10 can include a valve 15 housed within or coupled to the proximal end portion 14 that can be used to seal off the end of the lumen 36 to prevent blood from flowing outside the body.

Typically, although not necessarily, the perfusion device 10 is used to temporarily treat an injury to a blood vessel, such as a vessel rupture, until the patient can be transported to a medical facility where the blood vessel can be repaired. Thus, the perfusion device 10 can be implanted in a patient by, for example, emergency medical personnel in a battlefield or at the scene of an accident. In use, the perfusion device 10 is inserted into the patient's vasculature and advanced until the balloon 18 is in the vicinity of an injury to a blood vessel. FIG. 1, for example, illustrates an injury to the descending aorta A.

The perfusion device 10 can be inserted into a femoral artery and advanced through the patient's vasculature in a retrograde direction until the distal end of the balloon 18 is distal to the location of the injury. The proximal end portion 14 of the shaft 12 can serve as a handle for manipulating the perfusion device 10 and can remain outside the body when the balloon 18 is positioned at the desired deployment location. The balloon 18 can then be inflated, such as by activation of the syringe 22, causing the outer surface of the balloon 18 to contact and apply pressure to the inner wall of the aorta A on both sides of the injury I (i.e., upstream and downstream of the injury), as shown in FIG. 1. The balloon 18 creates a seal with the inner wall of the aorta A, causing blood to flow through into the distal end 24 of the shaft 12, through the lumen 36 of the shaft 12, and outwardly through the perfusion ports 26 (in the direction of arrows 28) within the confines of the vessel downstream of the injury, thereby bypassing the injury. The perfusion device 10 therefore protects against further bleeding while allowing for antegrade flow of blood to organs, extremities and collaterals to the spinal cord. In this manner, the perfusion device 10 can stabilize the patient during transport to a medical facility while minimizing the risk for organ failure, limb ischemia and paralysis. The perfusion device 10 can be removed from the body during surgery to repair the blood vessel by first deflating the balloon 18 and withdrawing the perfusion device 10 from the body.

In particular examples, the balloon 18 can be long enough to extend along substantially the entire descending thoracic aorta of the average human. For example, in certain examples, the balloon 18 can have a length L (FIG. 1) of at least 10 cm, and more desirably at least 15 cm. The balloon 18, when inflated, can have an outer diameter in the range of about 1.5 cm to about 2.5 cm, with about 2 cm being a specific example. The lumen of the shaft 12 can have diameter of about 4.0 mm or greater, or 4.3 mm in a specific example.

In some implementations, the perfusion device 10 can be inserted in the body using a conventional guidewire. For example, a guidewire can be inserted first into the patient's vasculature and advanced until the distal end of the guidewire is distal to the location of injury. The perfusion device 10 can then be inserted over the guidewire. The guidewire can extend through the main lumen of the shaft 12. Alternatively, the shaft 12 can have a separate guidewire lumen that extends from the distal end to the proximal end of the shaft.

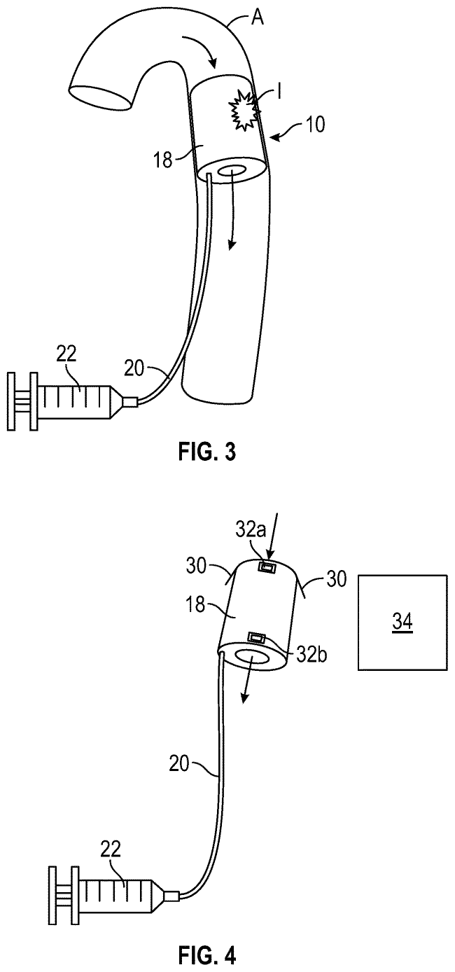

FIG. 3 shows an example of the perfusion device 10 without a shaft 12 shown deployed within the aorta A. In this example, the perfusion device can be introduced and delivered on a separate delivery catheter (not shown), and then subsequently retrieved and removed, such as during surgery to repair the aorta. For example, the balloon can be mounted in a deflated state on the distal end portion of a delivery catheter and then introduced into the patient's vasculature. The balloon 18 can be released from the delivery catheter upon its inflation, after which the delivery catheter can be withdrawn from the body. The balloon 18 in the example of FIG. 3 has an overall tubular shape defining an internal lumen or passageway through which blood can flow when inflated.

FIG. 4 shows another example of the perfusion device 10 having one or more anchors 30 mounted on the balloon 18. The anchors 30 are positioned to engage the vessel wall to assist in anchoring the balloon 18 in place within the vessel against blood pressure. The anchors 30 can comprise barbs that are configured to penetrate the surrounding tissue when the balloon is inflated. The anchors 30 can be made of any of various suitable biocompatible metals or polymeric materials. In one specific implementation, the anchors 30 can be made of a shape-memory, self-expanding material, such as Nitinol, and can be configured to expand radially from a stowed position for delivery to a deployed position extending away from the balloon for engaging the vessel wall.

As shown in FIG. 4, the perfusion device 10 can also include one or more position markers 32a, 32b that are detectable outside of the body to assist in positioning the balloon 18 relative to the vessel injury. In the illustrated example, the device is shown as having a single distal marker 32a mounted at the distal end of the balloon and a single proximal marker 32b mounted at the proximal end of the balloon. However, a greater or fewer number of markers can be used. For example, a plurality of markers can be spaced circumferentially around each of the distal and proximal ends of the balloon. It should be understood that the anchors 30 and/or the markers 32 also can be implemented in the example shown in FIGS. 1 and 2. Also, the position markers 32 can be mounted at other convenient locations on the perfusion device. For example, a perfusion device can include one or more position markers 32 (for example, RFID tags 32) mounted on the shaft 12 (e.g., a distal position marker mounted on the shaft 12 distal to the balloon 18 and a proximal position marker mounted on the shaft 12 proximal to the balloon 18).

In some examples, the position markers 32 can comprise magnets or magnetic material. In applications where fluoroscopy is available, the position markers 32a, 32b can be any of various radiopaque materials known in the art, including any suitable biocompatible metals or alloys (e.g., stainless steel). In such cases, the balloon 18 can be positioned relative to the injury under the guidance of a fluoroscope. Of course, fluoroscopy typically is not available where injuries occur (e.g., in a battlefield).

Hence, in particular examples, the position markers 32a, 32b can comprise passive or active emitters that can emit electromagnetic waves through the body and a corresponding external detector or monitor 34 (FIG. 4) can be used to receive the electromagnetic waves from the emitters and provide visual and/or audible feedback to a user indicating the position of the markers inside the body. In particular examples, for example, the position markers can be emitters that can emit radiofrequency waves, such as radiofrequency identification (RFID) tags. The position markers can be, for example, RFID microsensors or microsensors that are also configured to measure one or more hemodynamic or other physiological parameters of the patient, such as blood pressure and heart rate. In alternative examples, the perfusion device 10 includes one or more position sensors and one or more additional separate sensors that are configured to measure one or more physiological parameters of the patient. The monitor 34 desirably is a hand held unit and is powered by batteries or another lightweight, portable power supply to facilitate use in the field.

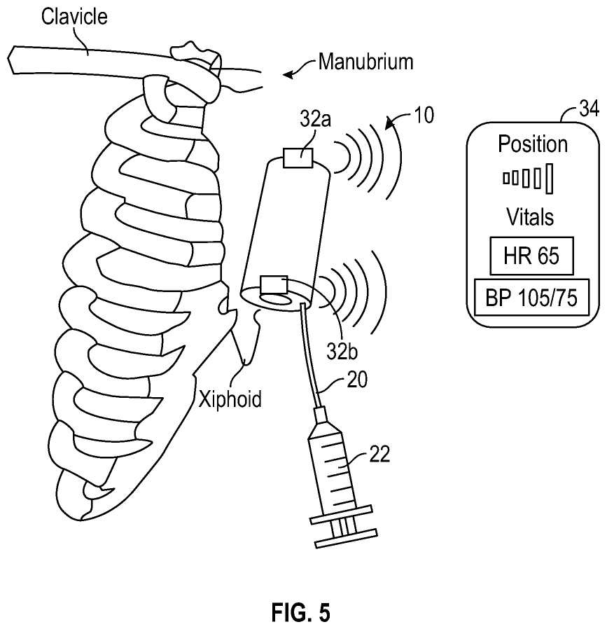

In the field (e.g., the combat theater), it is expected that placement typically will be performed under adverse conditions and by persons without advanced vascular experience. Accordingly, the emitters can allow for a rapid positioning between externally visible anatomic landmarks. As depicted in FIG. 5, for example, shows the placement of the perfusion device 10 at a location within the descending aorta between the xiphoid process and the manubrium. The xiphoid process can be used as a landmark for the celiac artery and the manubrium can be used as a landmark for the subclavian artery. Using these external bony landmarks, the user can position the distal end of the balloon 18 downstream of the subclavian artery and the proximal end of the balloon 18 upstream of the celiac artery to avoid obstructing these arteries.

The operating frequencies of the RFID tags 32 and the monitor 34 can be selected for detecting radiofrequency waves within several centimeters from the source of the waves. For example, frequencies in a low range (LF, 125-135 kHz) or a high range (HF, 13.56 MHz) can be used for communication within several centimeter separations. Detection of the RFID tags within the body can be accomplished in either a passive communication mode (the monitor 34 sends a carrier signal that is received and modulated by an RFID tag 32, which acts as a transponder and sends an identifying signal back to the detector) or in an active mode (both the monitor 34 and RFID tags 32 generate their own fields). In either case, the signal strength read by the monitor 34 is a function of the distance between the detector and a tag. When the monitor 34 is directly over an RFID tag 32, the signal strength is maximized, thereby enabling the user to determine the location of the tag with respect to external body landmarks.

A high frequency (HF) tag is advantageous in that it requires only a few wire turns as compared to a low frequency (LF) tag, which typically requires a hundred or more turns, resulting in a large axial dimension. Thus, the antenna pattern for a HF tag can be formed (printed) on a planar substrate, or directly on the balloon 18, using MEMS technology. In particular examples, an RFID tag 32 can comprise the control circuit of a commercially available RFID tag chip (e.g., a model NTAG203 from NXP Semiconductors) electrically connected to an antenna formed on the balloon 18 or on a separate layer mounted on the balloon.

As noted above, the perfusion device 10 can include one or more physiological sensors, such as a wireless blood pressure sensor. The blood pressure sensor desirably can detect blood pressure in a range of 0-150 mm Hg (0-20 kPa). In certain examples, the blood pressure sensor can comprise a pressure sensing device that measures the deflection of a diaphragm using resistive, capacitive or inductive methods. Moreover, the blood pressure sensor can be integrated in an RFID tag and can be mounted or formed on a component of the perfusion device 10, for example, on the balloon 18. The sensor can comprise a radiofrequency LC circuit comprising a capacitive pressure sensor that serves as a variable capacitor. In use, a change in pressure mediates a change in the resonant frequency of the sensor and is transmitted by RF signals to a monitor 34 held near the body.

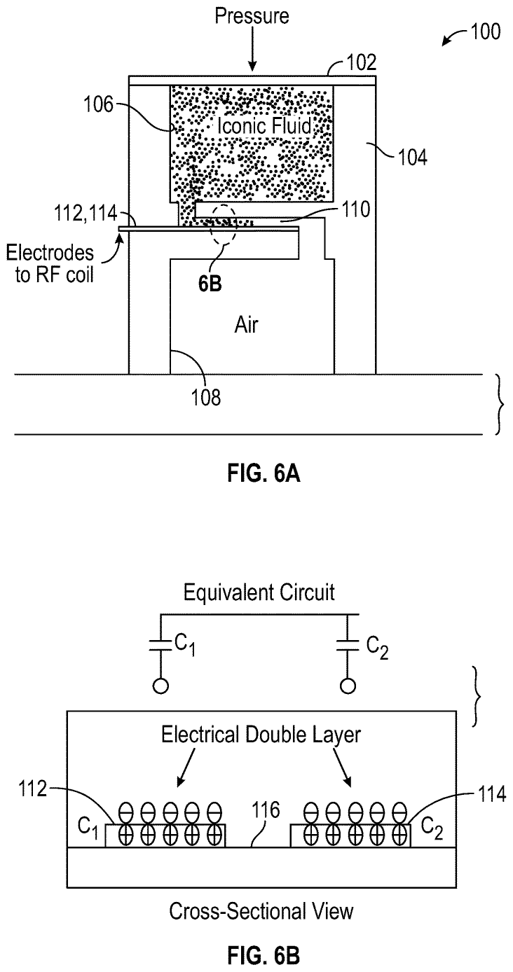

FIGS. 6A and 6B schematically illustrate the configuration and operation of a blood pressure sensor 100, according to one specific implementation. The blood pressure sensor 100 comprises a flexible diaphragm 102 mounted to a sensor body 104, which defines a first fluid chamber 106, a second fluid chamber 108, and a microchannel 110 extending between and being in fluid communication with the chambers 106, 108. The first fluid chamber 106 can be filled with a conductive (ionic) liquid and has an upper opening sealed by the diaphragm 102. The second fluid chamber 108 can be filled with a suitable pressurized gas, such as air. An inlet of the microchannel 110 is open to the first fluid chamber 106 and an outlet of the microchannel 110 is open to the second chamber 108 so as to allow the conductive fluid to flow into the microchannel upon application of pressure on the diaphragm 102. A surface of the microchannel 110 is formed with two spaced apart electrodes 112, 114 (e.g., indium tin oxide electrodes) defining a gap 116 therebetween extending lengthwise of the microchannel from the inlet to the outlet. The electrodes 112, 114 can be electrically connected to a radiofrequency (RF) coil of the sensor.

When the blood pressure sensor 100 is implanted in the body, the diaphragm 102 is exposed to and arranged in parallel to the blood flow so that it deflects under the static pressure of the blood flow and forces the conductive fluid in the first fluid chamber 106 to flow into the microchannel 110. As the conductive fluid flows into the microchannel, the interfacial areas between the electrodes 112, 114 and fluid increase and so do the capacitances between the two electrodes. The equivalent circuit between the electrodes is shown in FIG. 6B. Here, electrical double layers, which spontaneously form on the electrode surfaces as the conductive fluid flows along the length of the microchannel, serve as capacitors. The conductive liquid electrically connects the two double layers serving as an electrode for each double layer capacitor. In general, the thickness of the electrical double layers is in the nanometer range, which means that the capacitances are much higher than those found in other conventional or microscale capacitors since the capacitance is inversely proportional to the separation between conductors. This results in extremely high sensitivity in the present pressure sensing. The span of the capacitance can be controlled by adjusting the microchannel dimensions. Since the capacitance is proportional to how far the fluid flows into the microchannel, decreasing the height of the microchannel will provide a larger change in the interfacial area and thus capacitance for a given displaced fluid volume. At the same time, the height of the microchannel should be sufficient to minimize pressure drop, so as not to compromise the response time of the sensor. The compressed gas in the second chamber 108 acts as a buffer, allowing the conductive fluid to easily move back and forth in the microchannel as the external pressure on the diaphragm changes. The electrodes 112, 114 serve as the terminals to a wireless LC circuit, which can include an inductive coil printed on a surface of the sensor.

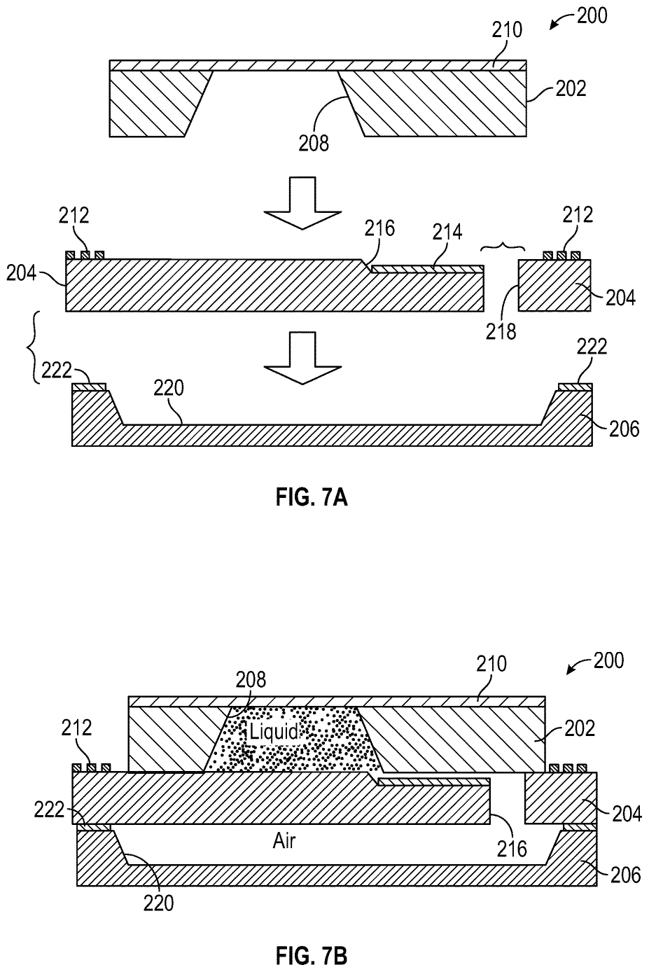

The blood pressure sensor can be made using micro-electromechanical (MEMS) fabrication techniques. For example, referring to FIGS. 7A and 7B, a blood pressure sensor 200 comprises an upper, first layer 202, an intermediate, second layer 204, and a lower, third layer 206. The layers 202, 204, 206 can be fabricated separately and subsequently assembled and secured to each other as shown in FIG. 7B. The first layer 202 can be formed from a silicon substrate having a silicon nitride membrane 210 (a Si₃N₄ membrane) on an upper surface thereof. Backside KOH etching can be used to form a chamber 208 that is open to the lower surface of the first layer 202. The membrane 210 serves as the diaphragm of the sensor.

The intermediate layer 204 can be formed from a glass substrate and can serve as a support for an antenna 212 and electrodes 214. Using a wet etching method, a recessed surface for a microchannel 216 can be formed in the glass substrate, followed by depositing and patterning of the electrodes 214 on the recessed surface. An aperture or hole 218 in the intermediate layer 204 can be made by drilling. A suitable metal can be deposited in a spiral pattern along the outer edge of the upper surface of the intermediate layer 204 to form the coils of the antenna. Both terminals of the antenna coils can be electrically connected to the electrodes in the microchannel, such as by respective traces on the intermediate layer 204. In order to minimize the ohmic resistance of the antenna, electroplating can be used for depositing the antenna.

Another glass plate can be used to form the third layer 206, which can be wet-etched to form a lower chamber 220. The three layers 202, 204, 206 can be assembled by bonding the first layer 202 to the intermediate layer 204 using, for example, anodic bonding, after which the chamber 208 can be filled with an ionic liquid. The third layer 206 can then be joined to the lower surface of the intermediate layer 204 using, for example, a suitable adhesive 222.

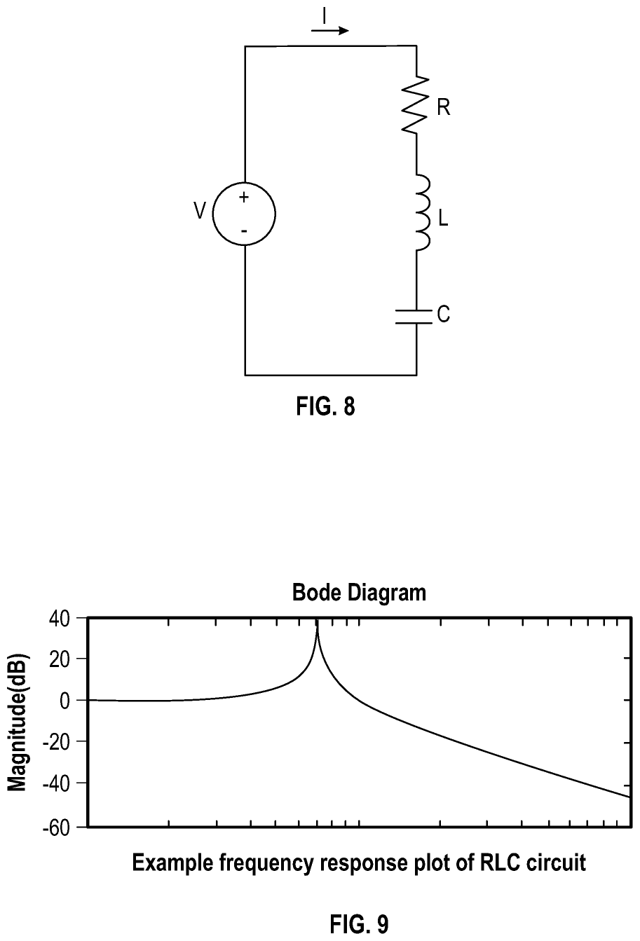

The sensor can be treated as the capacitive element of an L-C oscillator circuit, which enables its use as a passive device. A simple RLC circuit is depicted in FIG. 8. The voltage source is coupled with the inductor as the circuit receives energy from the external transmitter (e.g., monitor 34), the capacitor is related to the transducer, and the resistance (usually low) is inherent in the device. The governing equation for this circuit is

$$\frac{{d}^{2} i ( t )}{d {t}^{2}} + \frac{R}{L} \frac{d i ( t )}{d t} + \frac{1}{L C} i ( t ) = 0$$

As the system is excited from an external source, the current oscillates at the natural frequency of the system, \sqrt{1/LC}. Changing the capacitance or inductance changes the frequency, which can be detected when an inductively-coupled signal is received from the device. An example of the frequency response plot for an RLC circuit is shown in FIG. 9. For a high frequency system, this can be done repeatedly and continuously to monitor the changes in the sensor as pressure changes, or alternatively, transient behavior of the sensor can be monitored as opposed to the steady state behavior shown in FIG. 9. Given the short duration of use in certain applications, an active sensor and circuit design can be employed, which can improve time response (to capture continuous pressure measurements) and signal strength. For example, a capacitive bridge measurement technique can be employed or the change in capacitance can be directly transmitted rather than relying on the incoming energy from the monitor.

Thus, the blood pressure sensor can translate changes in pressure into resonant radiofrequency signals that can be detected by the external monitor 34. The monitor 34 can be programmed with software that processes the received signals and generates dynamic and physiologic blood pressure and heart rate readings. As shown in FIG. 5, the monitor 35 can have a visual display that displays the patient's physiological characteristics being monitored, as well the position of one of the position markers. The monitor can include a suitable microprocessor that can be programmed with software. In some examples, the monitor 34 can be a portable computer, such as a tablet computer, a smart phone, or a laptop computer.

In particular examples, a sensor assembly can comprise a position sensor (e.g., an RFID tag) and a blood pressure sensor that are electrically connected to a common antenna (e.g., antenna 212). Where a common antenna is used, the blood pressure signal can be used as the locating signal for position sensor; in other words, the position of the monitor 34 where the strongest blood pressure signal is detected is related to the position of the sensor in the body. If the device is active, the blood pressure sensor can be switched in and out of the antenna circuit, allowing the one antenna to function with both the position sensor and the blood pressure sensor. In an alternative example, a sensor assembly can comprise a position sensor (e.g., an RFID tag) having a first antenna and a blood pressure sensor having a second antenna, wherein the first and second antennas are physically and electrically separated, such as by forming the antennas on separate layers of the device or by forming one antenna coil concentrically within another antenna coil.

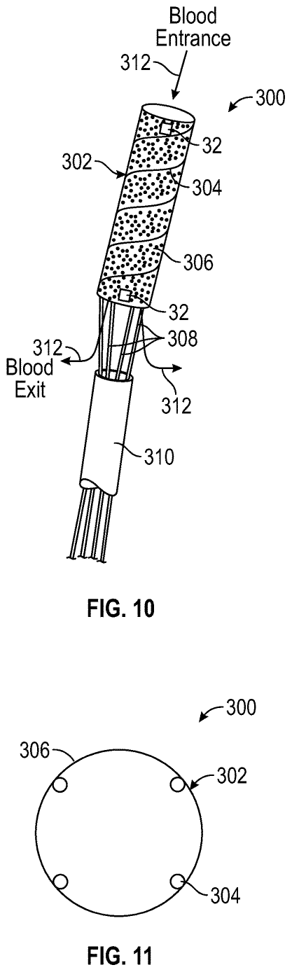

FIGS. 10 and 11 are, respectively, perspective and cross-sectional views of a perfusion device 300, according to another example. The perfusion device 300 comprises an expandable sealing member main body 302 comprising a self-expanding stent, or frame, 304 and a blood-impermeable tubular cover, liner or sleeve 306 supported on and covering the stent 304. The cover 306 can comprise any of various biocompatible fabrics, such as fabrics formed from polyethylene terephthalate (PET), polytetrafluoroethylene (PTFE), polyester urethane urea (PEUU), poly (carbonate urethane) urea (PCUU) or polyurethane fibers, or other types of synthetic fibers. The cover 306 alternatively can be a non-woven sheet of material made of any of these synthetic materials. Still alternatively, the cover 306 can be made of a blood-impermeable natural tissue, such as pericardium, or a thin metal film (e.g., Nitinol). The cover 306 can be secured to the stent 304 using suitable techniques, such as sutures, welding, or an adhesive. The cover 306 is shown as being mounted on the outside of the stent 304, but can be mounted on the inside of the stent in alternative examples.

The stent 304 can be a spiral wire as shown but can have configurations as well, such as a lattice or mesh type configuration similar to a coronary stent. The stent 304 can be made of Nitinol, stainless steel, cobalt chromium alloy or various other suitable materials. The perfusion device 300 can further include a plurality of rods or wires 308, the distal ends of which are connected to the main body 302. The wires 308 are long enough to extend out of the patient's body such that the proximal end portions of the wires can be manipulated by the user by application of pushing or pulling forces on the wires. In this regard, the proximal end portions of the wires 308 can be connected to a handle to facilitate insertion and withdrawal of the perfusion device from the patient's body. The perfusion device 300 can be used with an introducer sheath 310 which facilitates insertion of the perfusion device into the patient's vasculature and subsequent withdrawal of the device.

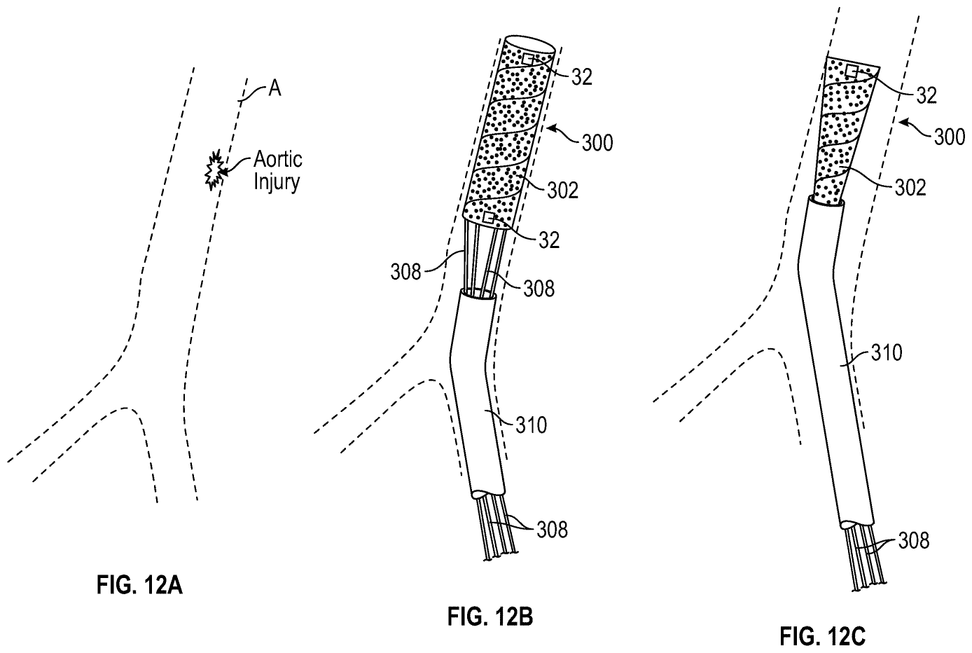

FIGS. 12A-12C illustrate use of the perfusion device 300 to treat a rupture of the descending aorta A, illustrated in FIG. 12A. As shown in FIG. 12B, the introducer sheath 310 is first inserted into the patient's vasculature, such as via a femoral artery of the patient. The introducer sheath 310 can have a length sufficient to extend to a location in the descending aorta while a proximal end portion (not shown) remains outside the body. The perfusion device 300 can then be inserted through the introducer sheath 310 and into the descending aorta until the main body 302 extends over and seals the ruptured portion of the aorta. As the main body 302 merges from the distal opening of the introducer sheath 310, it expands to its functional size contacting the inner wall of the aorta. The main body 302 can include one or more position markers 32 (e.g., RFID tags) as described above to help position the main body within the aorta. Once implanted, blood is caused to flow into the distal end of the main body, through the lumen of the main body, and outwardly through the proximal opening of the main body (in the direction of arrows 312 in FIG. 10), thereby bypassing the ruptured portion of the vessel. The patient can then be transported to a medical facility for surgery to repair the ruptured vessel.

As shown in FIG. 12C, the perfusion device 300 can be removed from the patient by retracting the wires 308 proximally and/or pushing the introducer sheath 310 distally to pull the main body 302 back into the sheath. Relative movement between the wires and the sheath causes the sheath to apply a radial force against the wires, forcing the wires to collapse radially, which in turn collapses the proximal end of the main body 302 enough to be pulled through the distal opening of the sheath. Further retraction of the wires pulls the main body back through the sheath and out of the patient's body.

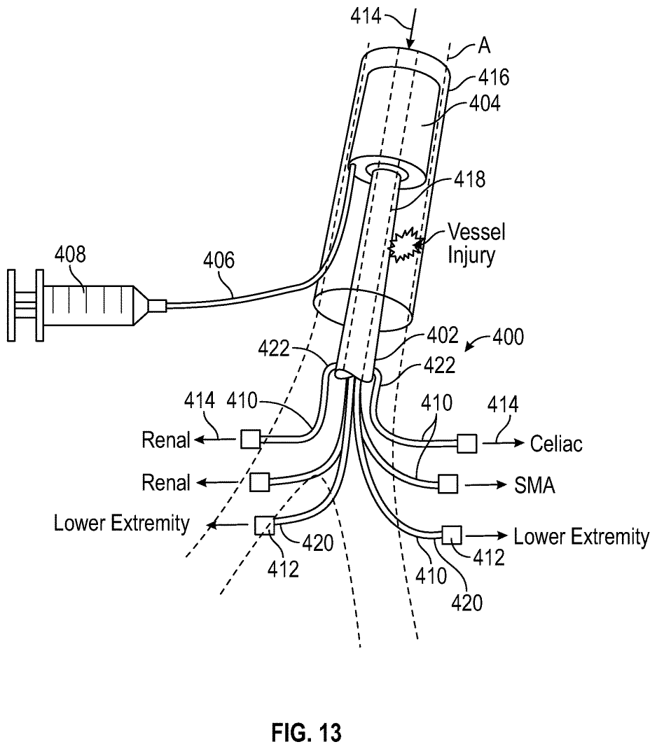

FIG. 13 shows a perfusion device 400, according to another example. The perfusion device 400 is configured to be implanted in the aorta and allow for the perfusion of blood from the descending aorta to downstream branch arteries during complex open aortic repair, in lieu of an aortic clamp or a left heart bypass. The perfusion device 400 can be used for open aortic repair in both military (for repairing trauma to the aorta) and civilian (for treating aneurysms) settings.

The perfusion device 400 in the illustrated example comprises an elongated shaft 402 and an inflatable balloon 404 mounted on the distal end portion of the shaft 402. The shaft 402 can extend from a distal end to a proximal end (not shown) outside the body. The perfusion device 400 can also include an inflation conduit 406 having a distal end fluidicly connected to the balloon 404 and a proximal end fluidicly connected to a syringe 408 or another source of an inflation fluid that is configured to pump the inflation fluid through the conduit and into the balloon.

The perfusion device 400 also comprises one or more secondary perfusion conduits, or cannulas, 410, which are in fluid communication with a lumen 418 of the shaft 402. The secondary fluid conduits 410 can comprise flexible tubular members, and can be made from any of various polymeric materials, such as polyurethane. The proximal end 420 of each secondary conduit 410 is configured to be positioned within a respective branch artery (e.g., a renal artery, the celiac artery, an artery feeding a lower extremity, or the superior mesenteric artery), as depicted in FIG. 13. In the illustrated example, the perfusion device includes six secondary conduits 410: two for renal artery perfusion, two for visceral branches (superior mesenteric and celiac arteries) and two for distal aortic (lower extremity and spinal cord) perfusion. In alternative examples, the perfusion device can include a greater or fewer number of secondary conduits, which can be positioned in other branch arteries.

To assist in retaining the proximal end portion of each secondary conduit within a branch artery, an inflatable balloon 412 can be mounted on the proximal end portion 420 of each conduit. Each balloon 412 can be fluidicly connected to separate source of an inflation fluid or to a common source (e.g., the syringe 408) by respective inflation-fluid conduits or a common inflation-fluid conduit.

The shaft 402 has a perfusion lumen 418 for a blood flow 414 that extends from a distal opening of the shaft (which is proximate the distal end of the balloon 404) to a location downstream of the balloon 404 where the lumen is in fluid communication with the secondary fluid conduits 410. For example, the distal ends 422 of the conduits 410 can extend through side ports in the shaft into the perfusion lumen. Thus, a pathway for blood extends from the distal end of the shaft, through the shaft lumen 418, and into and through each of the conduits 410. In alternative examples, the shaft 402 can be formed with multiple lumens extending from the distal end of the shaft to each of the secondary conduits 410. In other examples, one or more of the conduits 410 can extend from another conduit 410 to divert a portion of the blood flow 414 from one conduit to another. For example, the distal end 422 of a first conduit 410 can be fluidicly connected to a second conduit 410 at a location proximal to the distal end 422 of the second conduit, such that a portion of blood flowing into the second conduit is diverted into the first conduit.

Each balloon 404, 412 can include one or more anchors (e.g., anchors 30 shown in FIG. 4) to engage the inner walls of the vessels and/or can be formed with a relatively rough outer surface to increase the coefficient of friction of the balloon material against the vessel wall to increase resistance against balloon migration.

Also, instead of providing a separate inflation conduit 406, the shaft 402 can be formed with a separate inflation lumen that extends from the balloon 404 to a proximal end of the shaft outside the body. The proximal end of the inflation lumen can be fluidicly connected to a source of an inflation fluid (e.g., a syringe) to pump the inflation fluid through the inflation lumen and into the balloon 404. In addition, the inflation lumen in the shaft 402 can be in fluid communication with respective inflation lumens that are formed in and extend through each of the secondary conduits 410 to a respective balloon 412.

In use, such as during complex open aortic repair, the perfusion device 400 is placed in the aorta A such that the balloon 404 is upstream of a vessel injury or aneurysm to be repaired and the junction of the distal ends 422 of the secondary conduits 410 with the shaft 402 is downstream of the vessel injury or aneurysm. The proximal ends 420 of the secondary conduits 410 can be positioned in respective branch arteries and the balloons 412 can be inflated to help retain the proximal ends of the secondary conduits 410 in the branch arteries. The balloons 412 can be sized such that in their inflated state, the outer diameter of the balloons can contact and frictionally engage the inner walls of the branch arteries. Once the balloons 404, 412 are inflated, blood is caused to flow into the open distal end of the shaft 402, through the shaft lumen 418, through the secondary conduits 410 and into the branch arteries containing the secondary conduits, thereby bypassing the vessel injury or aneurysm. In certain examples, the shaft 402 can have side ports positioned proximally of the balloon (e.g., side ports 26 in FIG. 1) in communication with the lumen 418 to allow antegrade blood to flow outwardly through the side ports into aorta downstream of the vessel injury. The vessel injury or aneurysm can then be repaired using known surgical techniques, such as by suturing a prosthetic graft 416 over the injured/diseased portion of the aorta.

As noted above, the conventional clamp and sew approach used during open aortic repair increases the risk for organ failure, lower extremity ischemia and paraplegia. Conventional distal aortic perfusion by means of a left heart bypass entails increased complexity including additional perfusion equipment, expertise on the part of the surgeon and additional operative exposure. Further, the necessary equipment may not be available at many facilities. Advantageously, deployment of the perfusion device 400 is less complicated than performing a left heart bypass while allowing perfusion of the distal aorta and individual visceral vessels, thereby minimizing risk of organ failure, ischemia and paraplegia.

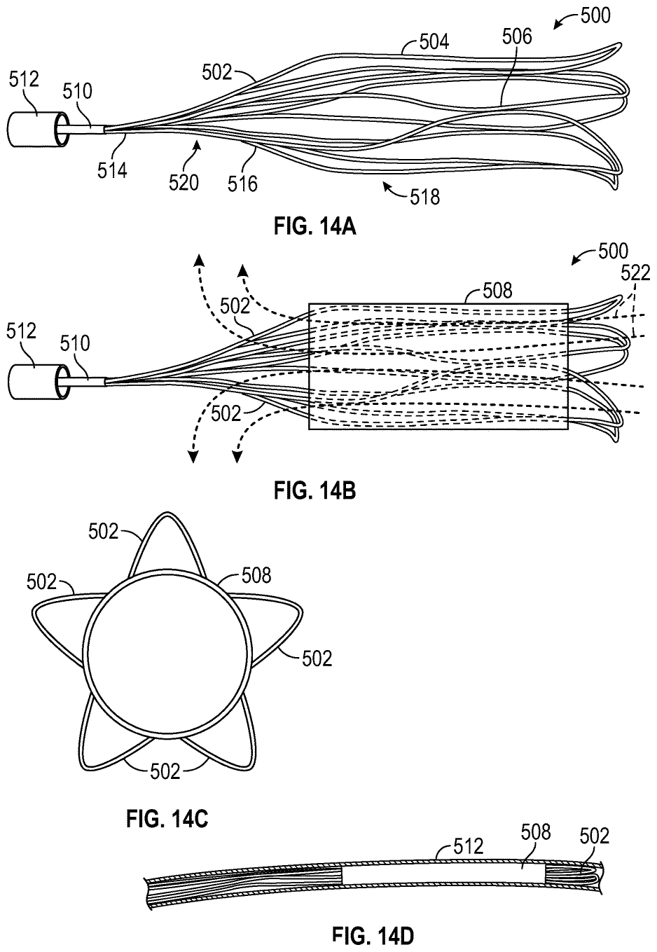

FIGS. 14A-14D show the distal end portion of a perfusion device 500, according to another example. The perfusion device 500 comprises a sealing member in the form of a self-expandable wire stent or frame 502 and a blood-impermeable cover 508 or (alternatively called a sleeve 508) mounted on the outside of the frame 502 (as shown in FIG. 14B). FIG. 14A shows the frame 502 without the cover 508 for purposes of illustration. The frame 502 in the illustrated example is formed from a plurality of petal-shaped wires 504. Each wire 504 in the illustrated example forms a longitudinally extending loop having a first end portion 514 and a second end portion 516 secured to the first end portion 514. The longitudinally extending loops can be circumferentially arranged and secured to each other along their adjacent edges at junctions 506. The frame 502 can have a generally cylindrical distal end portion 518 and a tapered proximal end portion 520 to facilitate recapture of the stent into a delivery sheath 512. The cover 508 can extend over and cover at least the majority of the length of the cylindrical distal end portion 518 as shown in FIG. 14B, but also can extend over and cover a portion or the entire length of the tapered proximal end portion 520.

The device 500 can further include a shaft 510, the distal end of which is fixedly secured to the proximal end portions 514 of the wires 504 of the frame. The shaft 510 has a length sufficient to extend through a patient's vasculature to position the sealing member at the location of an injury to a blood vessel. The proximal end of the shaft 510 can be coupled to a handle to facilitate advancement and retraction of the device within the patient's vasculature. Alternatively, the wires 504 can extend all the way to the handle outside the body without a separate shaft coupling the wires to the handle. The device can include one or more position markers (e.g., RFID tags) mounted at a convenient location, such as on the distal and proximal end portions of the cover 508.

In one specific implementation, the wires 504 of the frame can be made of a shape-memory material, such as Nitinol, but can be formed from other suitable materials, such as stainless steel, or a cobalt chromium alloy. In one specific implementation, the cover 508 can be a thin metal film (e.g., Nitinol) affixed to the wires of the frame, such as by welding. In other examples, the cover 508 can comprise any of various biocompatible fabrics, such as fabrics formed from polyethylene terephthalate (PET), polytetrafluoroethylene (PTFE), polyester urethane urea (PEUU), poly (carbonate urethane) urea (PCUU) or polyurethane fibers, or other types of synthetic fibers. The cover 508 alternatively can be a non-woven sheet of material made of any of these synthetic materials, or a blood-impermeable natural tissue, such as pericardium. In the illustrated example, the cover 508 is shown mounted to the outside of the frame, however, in alternative examples, the cover 508 can be mounted to the inside of the frame.

The frame 502 is configured to be self-expandable from a radially compressed or collapsed state (FIG. 14D) to a radially expanded, deployed state (FIG. 14B). The device 500 can further include a delivery sheath 512 that extends over the frame 502 and retains it in the radially collapsed state for delivery through the patient's vasculature.

The device 500 can be used to treat a ruptured blood vessel in the manner described above with reference to FIGS. 12A-12C. With the frame retained in a collapsed state within the delivery sheath 512, the perfusion device 500 can be inserted into a patient's vasculature (e.g., into a femoral artery) via an introducer sheath 310. The shaft 510 (or handle attached to the proximal end of the shaft) can be used to push the perfusion device 500 through the introducer sheath 310 and the patient's vasculature until the distal end portion of the perfusion device is in the vicinity of the ruptured portion of the blood vessel. To deploy the frame at the site of a vessel injury, the user can push the shaft 510 distally and/or retract the delivery sheath 512 proximally to advance the frame from the distal opening of the sheath, allowing the frame to self-expand such that the cover 508 forms a seal against the inner wall of the vessel. Once deployed, blood can flow through the lumen defined by the cover 508 in the direction indicated by arrows 522. After treatment of the blood vessel, the frame can be retracted back into the delivery sheath 512 and the perfusion device 500 can be removed from the patient's body.

### Magnetic Location Detection

Also disclosed herein are examples of a magnetic location detection system for identifying the position of an implanted device such as the perfusion devices 10, 300, and 400 described in greater detail above. It is to be understood that, while the more detailed discussion below is directed towards the use of the magnetic location device system for treating aortic injuries, it may also be used to treat caval (venous) injuries, or for guidance in other parts of the body, such as intracranial guidance, or guidance in the chest and/or abdomen.

Hemorrhage-type injuries represent a high fraction of battlefield injuries, and in many cases are potentially survivable. These injuries may be treated with the application of an aortic stent graft, but the placement of such devices may be challenging outside of a facility with fixed fluoroscopic equipment and other equipment required for precise positioning of the stent, within the vasculature of the patient. Such injuries, however, are likely to be sustained in locations (for example, on the battlefield) where such fixed equipment is unavailable. Such injuries may also be highly time sensitive. Aside from the hemodynamic effects of hemorrhage, rapid blood loss may cause coagulopathy, multi-organ failure, and conditions such as Systemic Inflammatory Response Syndrome. It is therefore desirable to rapidly stop or minimize blood loss with a treatment method so that the patient can be transported to a medical facility with the proper equipment for a long-term treatment to be employed.

Devices such as the perfusion devices 10, 300, and 400 described above may be suitable to rapidly stop or minimize the blood loss from such injuries, by deploying a stent to the site of the injury. However, effective use of such a perfusion stent relies on accurate and reliable placement at the site of the injury, and the emergency use of a perfusion stent is often done at a location that lacks sophisticated imaging equipment. If the stent is placed improperly, portions of the patient's vasculature can become blocked or impeded by the stent, which can cause lactate acidosis, sepsis, renal insufficiency, and even fatal complications. Therefore, there is a need for a robust, reliable, and portable system for accurately positioning a stent or other medical device within the vasculature of the patient.

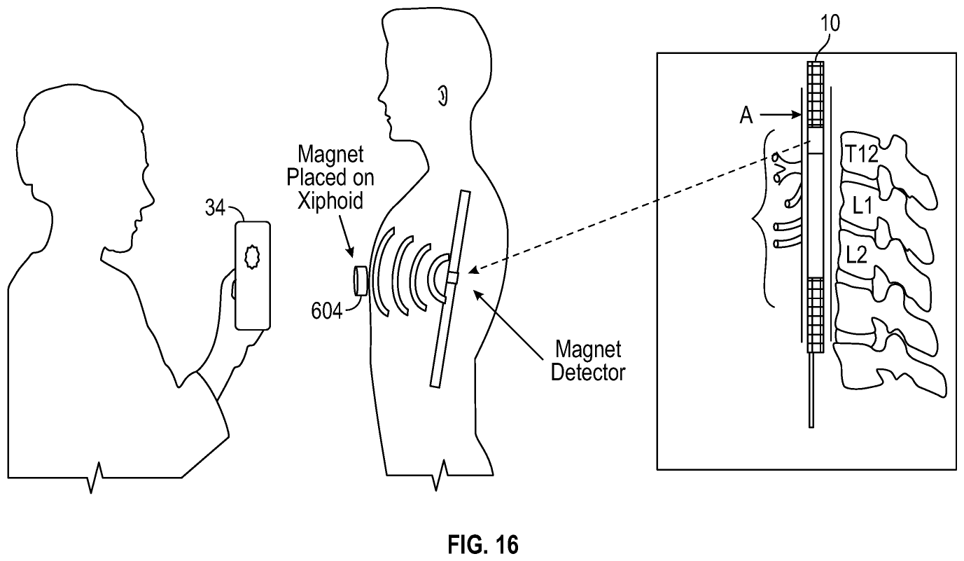

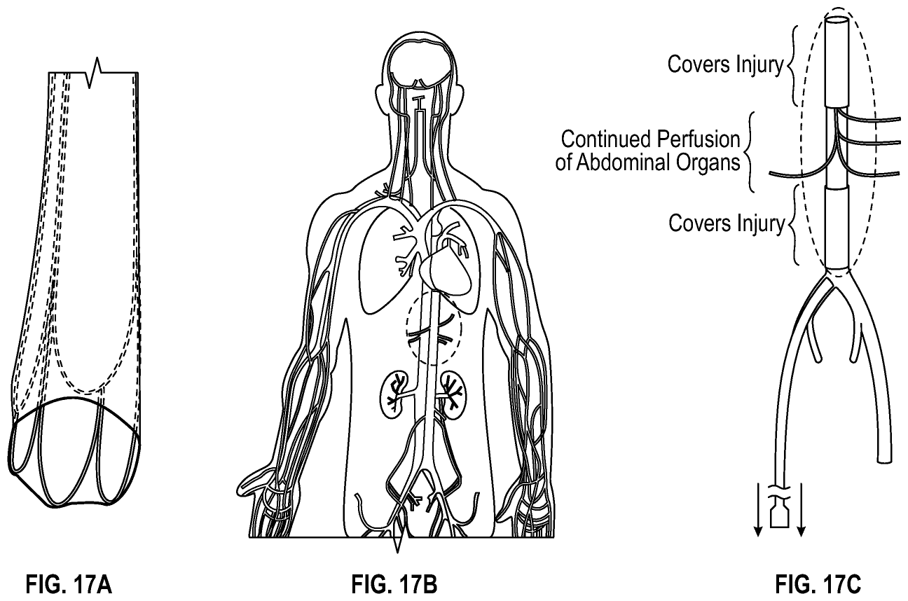

FIG. 16 illustrates how a perfusion stent, such as the perfusion device 10, is placed in the body when the wounded major artery is the celiac trunk, which contains branches of the hepatic, splenic, and left gastric arteries. These branches may become blocked by the stent, resulting in lactate acidosis, sepsis, renal insufficiency, and even fatal complications. To avoid occlusion where vital branch arteries are located, the stent contains two regions that are covered by a sealing material, such as PTFE, which effectively seals the artery at the wound, and an open section in which there is no sealing material, allowing blood flow to branching arteries, as shown in FIG. 17. During placement, the uncovered section is matched with the branch arteries to keep the branching routes open. For example, the celiac trunk generally lies within 1-3 cm of the xiphoid bone, which can be felt externally on the body. Therefore, in such examples, the xiphoid bone can be regarded as the landmark of the target location.

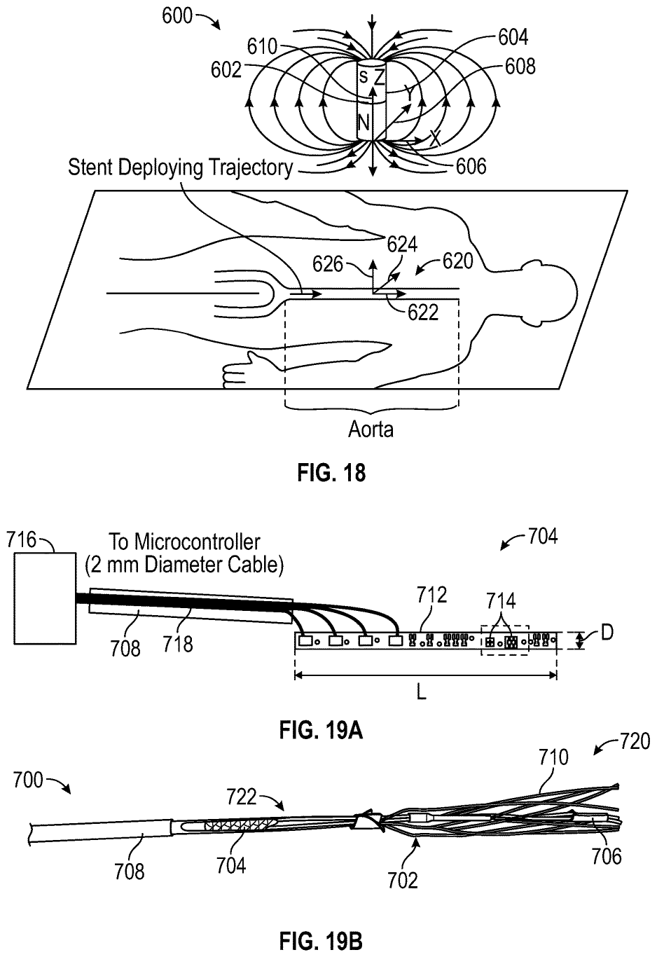

Generally, the location of an implanted device can be measured by generating a magnetic field of known shape and intensity and measuring that field at the location of the implanted device to determine the location of the device relative to the origin of the field, with relation to a known physical location on the patient's body to establish a three-dimensional magnetic coordinate system 600 with a known origin 602, as illustrated in FIG. 18. Generally, the physical reference location can be an external location, such as a bone (for example, the xiphoid bone as previously disclosed) that acts as a reference point and/or surrogate location for the damaged vessel. If the magnet coordinate system is aligned with and at a known location relative to the body coordinate system, then one can transfer a known location in the magnet frame to the body frame. This relative alignment and positioning is done by first placing the magnet on or near the body in a known position and orientation relative to an appropriate landmark, such as a bone of the body.

The magnetic coordinate system 600 with a known origin 602 can be derived from the theoretical EMF reference around a reference magnet 604. When the magnet 604 has a simple geometry, such as the cylindrical geometry indicated in FIG. 18, the origin 602 of the coordinate system 600 can be approximated as being located at the center of the magnet 604. The magnetic coordinate system comprises a first magnetic axis 606, a second magnetic axis 608 perpendicular to the first magnetic axis 606, and a third magnetic axis 610 perpendicular to the first magnetic axis 606 and the second magnetic axis 608.

In some examples, particularly those using a non-permanent magnet, such as an electromagnet, a magnetic baseline for the patient and the surrounding environment can also be established by cycling the magnet between an active state and a non-active state. In the active state, the electromagnet can produce an active magnetic field and in the non-active state, the electromagnet may not produce a magnetic field (however, importantly, environmental sources of an electromagnetic field may still be present). By alternating the magnet 604 between an active state and a non-active state, interactions between the magnetic field and magnetically-responsive externalities, such as ferrous metal, electronics, motors, etc., in the magnetic field generated by the magnet 604 can be measured. Advantageously, this allows for a background or baseline to be established that accounts for any distortions to the magnetic field caused by local conditions, such as magnetic noise and/or interference. These local conditions can then be subtracted from the measured magnetic field during stent deployment to facilitate measurements in a theoretical field that more closely approximates a “white balance” condition (i.e., a magnetic field condition with no interference from local conditions).

In examples using electromagnets, heat may also be a concern. Electromagnets generate heat when in the active state, and this heat can reduce the accuracy of measurements of the magnetic field. Furthermore, a hot magnet may cause patient discomfort or even injury. By cycling the magnet 604 between the active state and the non-active state, the magnet can be given time between active state periods during which it is not generating heat and can be allowed to cool.

In some examples, a plurality of reference magnets 604 can be placed at a corresponding plurality of reference locations on the patient's body. The relative locations of the plurality of reference magnets 604 to each other and to the corresponding reference locations on the patient's body in such examples may be known, and used to generate a composite reference magnetic field on which to base the magnetic coordinate system 600. Advantageously, the use of a plurality of reference magnets 604 can improve the accuracy and robustness of the measurements taken within the field, and allow for superior isolation and correction of externalities and/or disturbances to the magnetic field.

The magnetic coordinate system 600 can also be aligned with a three-dimensional body coordinate system 620 comprising a first axis 622, a second axis 624 perpendicular to the first axis 622, and a third axis 626 perpendicular to the first axis 622 and the second axis 624. With continued reference to FIG. 18, the body coordinate system 620 according to one example implantation method can have a first axis 622 aligned with the aorta of the patient (in other words, aligned along the length of the torso), a second axis 624 transverse to the aorta and in the horizontal plane of the patient's body, and a vertically-oriented third axis transverse to both the first axis and the second axis and substantially normal to the patient's body (i.e., aligned with gravity when the patient is lying down as shown in FIG. 18). The origin of the body coordinate system can be assumed to be in the center of the aorta, directly under the xiphoid, though in general it could be located at any point relative to any bony landmark.

In one example, for treating an injury to the aorta of a patient, a cylindrical magnet 604 can be used to generate the magnetic field that provides the basis for the magnetic coordinate system 600, using the xiphoid bone of the patient, which lies along the centerline of the aorta, as a reference, as shown in FIG. 18. For a cylindrical magnet, the radial component of the EMF is isotropic. Therefore, when the cylindrical magnet 604 is placed on or adjacent to the xiphoid bone of the patient, the first magnetic axis 606 of the magnetic coordinate system 600 is automatically aligned to be parallel with the first axis 622 of the body coordinate system 620, and the second magnetic axis 608 of the magnetic coordinate system 600 is automatically aligned to be parallel to the second axis 624 of the body coordinate system 620. In addition, the third magnetic axis 610 of the magnetic coordinate system 600 is aligned vertically in space and is parallel to the third axis 626 of the body coordinate system 620.

Thereafter, a medical assembly, such as the medical assembly 700 shown in FIG. 19B, can be inserted into the vasculature of the patient. As shown in FIG. 19B, the medical assembly 700 can comprise a stent 702, a detector 704, a guidewire 706, and a sheath 708. The stent 702 can comprise a radially expandable frame 710 and a cover, such as cover 306 discussed in greater detail above and illustrated in FIG. 10, disposed either around or within the frame 710.

The detector 704 is illustrated in greater detail in FIG. 19A, and can comprise a printed circuit board 712 with one or more sensors 714 fixedly attached thereto. The sensors 714 can include a triple-axis magnetometer, and in some examples can also include an accelerometer, and/or a gyroscope, and can be configured to collect environmental data within the patient. The detector 704 can also comprise a microcontroller 716 and one or more cables 718 to transmit signals between the sensors 714 and the microcontroller 716. In alternative examples, the detector 704 can include a wireless transmission device in addition to or in lieu of the cables 718, to enable signals from the sensors 714 to be sent wirelessly to the microcontroller 716.

In some examples, it may be desirable to minimize the size of the detector. While the detector 704 must be large enough to accommodate the sensors 714, the cables 718, and any other components such as a wireless transmission device, the diameter of the detector may influence the minimum diameter of the medical assembly 700 while it is compressed in the sheath 708. For this reason, it is desirable to keep the diameter of the detector 704 below 5 mm, and preferably no greater than 2-3 mm. In one particular example illustrated in FIG. 19A, the detector has an overall length, L, of 30 mm and an overall diameter, D, of 1.8 mm.

In some examples, the sensors 714 of the detector 704 can comprise a plurality of magnetometers, each placed at different locations relative to the stent 702. Because the locations of the magnetometers are known relative to the stent, the detector 704 can be configured to form a composite magnetic measurement to determine the position of the medical assembly 700 within the body of the patient. Advantageously, when the detector 704 comprises multiple magnetometers, determination of the location of the medical assembly 700 within the body of the patient may be less sensitive to individual externalities that may distort the magnetic field and/or the measurements taken by each individual magnetometer, thereby improving the accuracy and reliability of the measurements of the location of the medical assembly 700. The stent 702 and the detector 704 can, as shown in FIG. 19B, be mounted to the guidewire 706, which extends through the sheath and has a distal end portion 720 positioned away from the sheath 708 and a proximal end portion 722 positioned adjacent to the sheath 708. In some examples, the detector 704 can be positioned alongside or within the stent 702 on the guidewire 706, but in other examples, the detector 704 can be placed closer to the distal end portion 720 or to the proximal end portion 722 of the guidewire than the stent 702. The position of the detector 704 relative to the stent 702 is recorded.

The medical assembly 700 can be inserted into the vasculature of the patient and advanced along the guidewire 706 towards the desired implantation site with the stent 702 and the detector 704 retained within the sheath 708. As the medical assembly 700 advances through the vasculature of the patient, the three-dimensional magnetometer can measure the strength and vector of the local magnetic field. In some examples, many measurements may be taken to verify the position of the detector and improve the accuracy of position measurements. Because the magnetic field is known and recorded, the measurement of the three-dimensional magnetometer can be matched to a corresponding known strength and vector of the known magnetic field to determine the position of the detector relative to the origin point of the magnetic field (i.e., the magnet) in terms of magnetic coordinate system 600. Since the magnetic coordinate system can be mapped onto the body coordinate system 620 with a known offset, once the location of the sensor is known in the magnetic coordinate system 600, the corresponding location of the sensor in the body coordinate system 620 may be calculated. In turn, because the location of the detector 704 relative to the stent 702 is also known, the position of the stent 702 within the vasculature of the patient may also be calculated, using the body coordinate system 620 as a reference.

Because the location of the can be assumed to be within the length of the aorta, and because a known reference point (for instance, the xiphoid bone) relative to the aorta has been identified, the stent 702 can thereby be accurately delivered to cover the full length of the aorta, except for the branching vessels. Advantageously, this method does not require conventional fixed fluoroscopic imaging equipment to accurately deploy the stent along the injured portion of the patient's vasculature, and therefore may be suitable for use when injuries are sustained in a location (for instance, a battlefield) where such equipment is not available.

When the medical assembly has been positioned at the desired implantation site, such as the location of the injury to the patient's vasculature, the stent 702 and the detector 704 can be deployed from the sheath 708. The stent can then be radially expanded until it abuts the internal diameter of the patient's vasculature at the location of the injury, sealing or substantially sealing against the injured portion of the patient's vasculature and stopping or reducing blood loss without obstructing the flow of blood through the patient's vasculature. In some examples, the radially expandable frame 710 of the stent 702 can be a self-expanding frame 710, as discussed in greater detail above. In other examples, the frame 710 can be configured to be mechanically expanded, for example, by inflating an inflatable balloon positioned radially inwards of the frame 710 to deform the frame radially outwards until the frame 710 has the desired diameter, as discussed for the perfusion devices described in greater detail above.

While the methods described above refer to the treatment of an injury to the celiac trunk, it is to be understood that the methods are also applicable to other injury locations, such as caval or venous injuries. Furthermore, the magnetic location detection system described above can be used in other applications, such as intracranial guidance, or the guidance of devices within other body cavities, such as the chest or abdomen. It is also to be understood that, while the xiphoid bone may be selected as the reference point when deploying a stent to treat an injury to the celiac trunk, other fixed locations, such as other bones, may be selected as reference points when treating applications to other parts of the body. It is also to be understood that the magnetic location detection system described above can be used with other implantable medical devices.

### EXAMPLES

### Example 1—Signal Detection

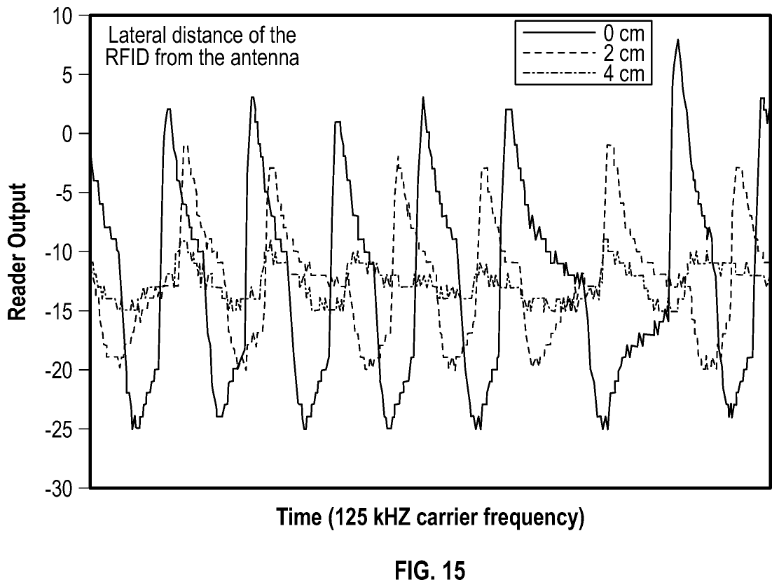

In one example, a commercially-available RFID, low frequency tag (2-mm diameter and 1-cm long, frequency 125 kHz) and monitor were used to simulate positioning of a perfusion device within the body. To simulate localization, a stack of paper and a plexiglass sheet (total 3 cm thick) were placed between the monitor antenna and RFID tag. The tag was placed at different distances from the center of the monitor antenna, which was measured by the ruler on the plexiglass. The signal output at the monitor vs. the lateral distance from the antenna center is shown in FIG. 15. All of the wave profiles were modulated according to the data stored in the tag. In particular, the amplitude of signal output was found to monotonically decrease as the distance increased. When the tag was placed right above the center of the antenna, the amplitude was maximized. In other words, the point of maximal signal indicates to the user the location of the radiofrequency tag in the body. From the data shown, the change in signal magnitude allows localization (positioning) of a perfusion device well within a +/−2 cm range.

The optimal detection distance in this example was 3 cm. The detection distance is closely related to the induced voltage in the tag antenna (V_{tag}) that has to be high enough to activate and energize the tag circuit. In inductive coupling in LF and HF tag communication, the tag voltage is calculated as follows:

$${V}_{tag} = 2 π fNQBS cosα ,$$

where f is the frequency of carrier signal, S the area of the tag coil, Q the quality factor of the resonant circuit, B the strength of the magnetic field at the tag, and α the angle of the magnetic field normal to the tag. Due to the size restriction in the tag, there is not much leeway for changing S and N for the fixed frequency. However, B can be relatively easily increased by changing the current and area of the monitor antenna. The increased voltage in the tag can allow for an increase in the detection distance for improved clinical performance. The angle of the magnetic field normal to the tag area (a) effects the detection distance, as the tag voltage is a cosine function of α In this example, α was set to 90 degrees so that the tag could be activated over a distance of at least 3 cm. However, the detection distance would be decreased if the angle α is not maintained during use.

Thus, in some examples, the perfusion device can include multiple RFID tags with different IDs spaced circumferentially around the balloon. The tags can be scanned individually, or several at a time, depending on circumstance. Among the multiple tags, the best-aligned tag provides a maximum output and thus maximum detection distance, which then can be used for subsequent positioning of the balloon.

### Example 2—Proof of Concept

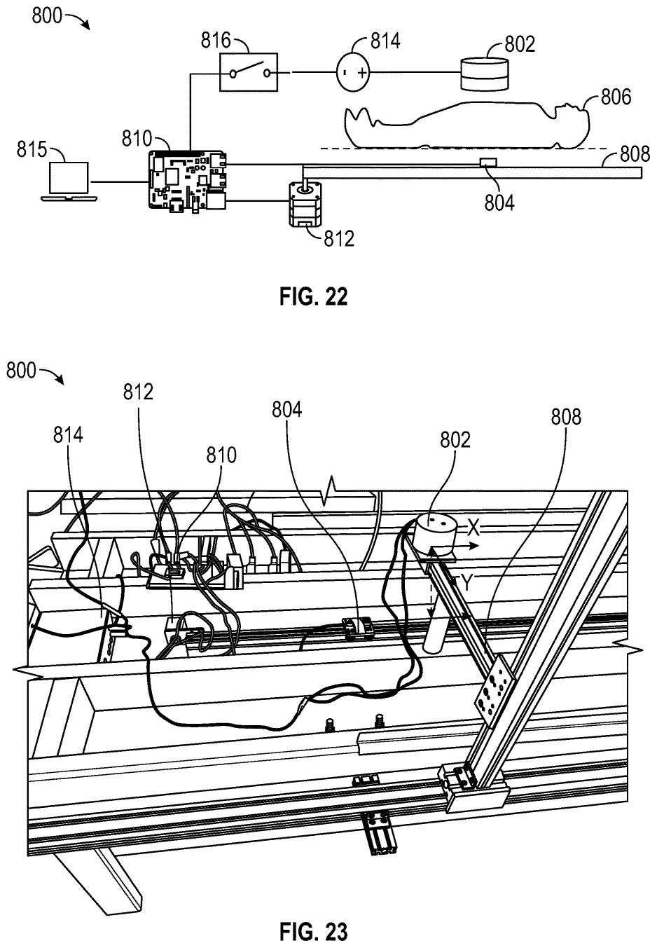

Testing has been completed to quantify the accuracy of the system and to demonstrate its feasibility for use in trauma applications. An example test assembly 800 is shown in FIG. 22. FIG. 22 shows a schematic of the test assembly 800 and testing conditions. The test assembly 800 comprises an electromagnet 802 and a detector 804. The electromagnet 802 and the detector 804 can be positioned to either side (for example, above and below) of a human subject 806. The detector 804 is mounted to a rail 808 which allows the detector to be moved along one axis relative to the electromagnet 802, to simulate the advancement of a prosthetic device, such as the perfusion device 10 described herein and shown in FIG. 1, through the aorta of the patient 806.

A control module 810 (controller) controls both a motor 812 (such as, for example, a stepper motor) and the activation of the electromagnet 802. The motor 812 can advance and retract the detector 804 along the rail 808 as described herein, in response to guidance commands from the control module 810. The electromagnet 802 can be alternated between an active state and an inactive state by toggling a power supply 814 between an on state and an off state, for example by toggling a relay 816 between an open and closed configuration.

Results, such as measurements of the magnetic field generated by the electromagnet 802 and measured by the detector 804 can be displayed externally to the patient 806 on a monitor 815.

FIG. 23 illustrates the apparatus used to complete the test of system accuracy. The stepper motor 812 is used to drive the detector 804 to known locations, which are used to check the accuracy of the measurement. The motor 812 drives the detector 804 to move on a straight rail 808, which simulates deployment in an artery, whose direction is aligned in parallel with first magnetic axis 606 (that is, the X axis) in the magnetic coordinate system 600. In this simplified condition, the coordinate change along the rail only occurs along the first magnetic axis 606 component, with the detector 804 being static along other two component axes 608 and 610. For the last step, the sensor is deployed within a water-filled plastic tube and covered by pork to simulate the in-vivo condition.

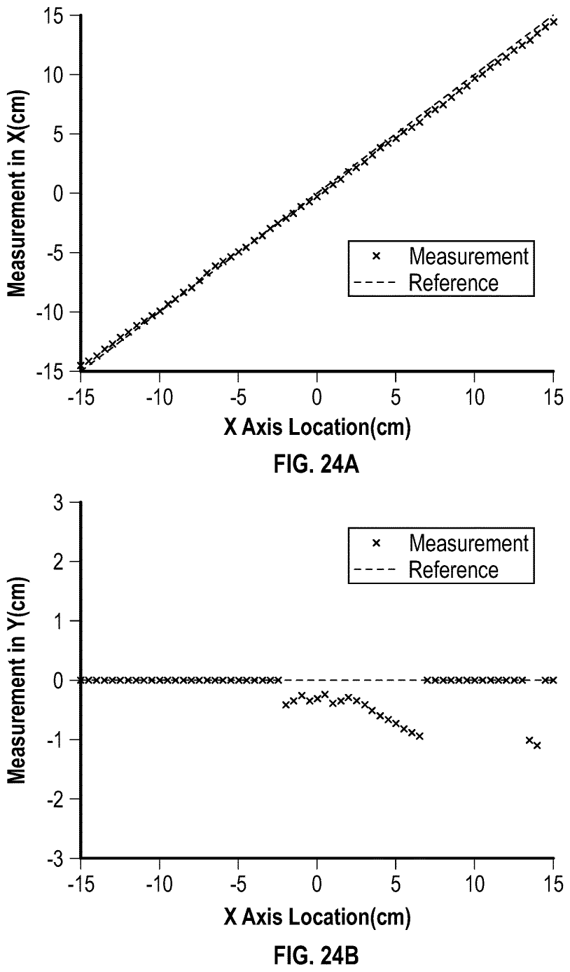

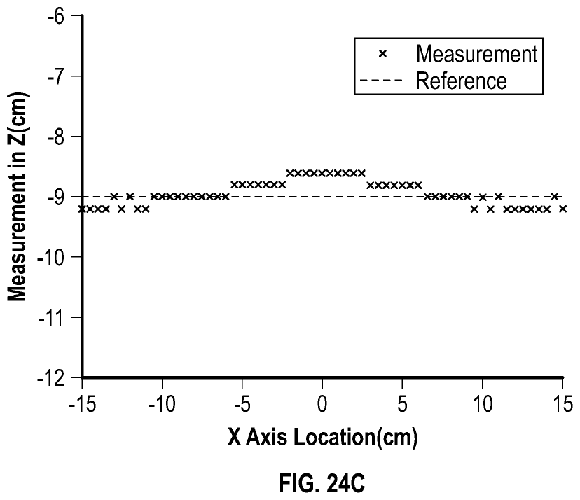

Sample results of the tests are shown in FIGS. 24A-24C. As shown in FIGS. 24A-24C, the system generally is accurate to within 1 cm, which is sufficient for the application of interest, and additional testing has been completed using live pigs to demonstrate that the accuracy holds to the level needed for stent placement in a human patient in a trauma situation. In one example, testing has been conducted on live pigs to simulate stent placement in the presence of adjacent metal to simulate the magnetic distortion expected from shrapnel. In another example, testing has been conducted on live pigs in conditions that simulate hemodynamic disturbances such as high blood pressure, low blood pressure, or accelerated heart rate, which a patient may be expected to experience during deployment of the stent. In a further example, testing has been conducted on live pigs while the pigs were subjected to environmental vibration to simulate conditions applicable to a patient being transported as the stent is deployed. It is expected that the system will retain sufficient accuracy to deploy the stent to the desired location despite substantial environmental disturbances and/or externalities, such as those experienced in emergent situations where shrapnel, unstable patients, and vibration related to patient transportation are expected. As described further herein, additional methods are used to improve accuracy of the device when conditions deviate from ideal, such as when there are external sources of magnetic interference.

### Example 3—Practical Applications

For persons injured on the battlefield, effective and prompt control over internal hemorrhage, particularly for major vessel trauma, is important for their survival. According to a research study, 20% of mortalities during a war happen before the soldiers can reach treatment facilities, and the major cause in this group is hemorrhage, which is over 50%. For the extremely dangerous condition in which the wound occurs in the aorta deep inside the torso, where hemostasis is hard to achieve externally with compression or a bandage, a retrievable rescue perfusion stent can be used. In one example, the cylindrical stent is covered with an impermeable PTFE layer. When deployed into the aorta, the stent expands to support the PTFE layer to cover the wound in the vessel, thereby creating an effective second interior artery wall, which has been shown to reduce blood loss by hemorrhage by 93% while the blood can still flow within. As an emergency-aid device for when the location of the wound cannot be accurately visualized, the stent may be designed to be long enough to cover a large portion of the artery. This design could cause side effects since all branch vessels along the artery are occluded by the stent. For example, when the wounded major artery is the celiac trunk, which contains branches of the hepatic, splenic, and left gastric arteries, these branches may become blocked by the stent, resulting in lactate acidosis, sepsis, renal insufficiency, and even fatal complications. To avoid occlusion where vital branch arteries are located, a portion of the aorta can be left uncovered by the PTFE layer. During placement, the uncovered part can be matched with the branch arteries to keep the branching routes open.

The celiac trunk generally lies within 1-3 cm of the xiphoid bone, which can be felt externally on the body. Therefore, the xiphoid bone can in some examples be regarded as the landmark of the target location. Location measurement of the device inside the body with respect to this landmark enables proper placement of the stent that leaves the branch vessels free for blood flow. This desired function of the stent is illustrated in FIGS. 17A-17C, which show the stent in the deployed configuration (FIG. 17A), the native vasculature of the patient (FIG. 17B), and the deployment of a stent over the aorta of the patient, leaving the branch vessels free of the stent members (FIG. 17C).

Current object locating techniques for surgical application can be achieved in several different ways. The first method is to use imaging of the body features inside the volume of interest in the body. In the case of detecting location of a stent or other foreign object, the images are analyzed to determine the object's location relative to anatomical features. Imaging modalities include fluoroscopy, computed tomography, magnetic resonance imaging, and ultrasound. Because of their clarity and reliability, image-based systems are widely implemented in hospitals. However, these systems typically rely on real-time imaging when used to track an object, and perhaps more importantly are not portable and are inconvenient to use, especially for emergent situations such as those on the battlefield. Therefore, portable systems that can be rapidly deployed have been developed as alternatives.

The second method of object tracking that fulfils the feasibility requirements is to measure or estimate the motion of the object as it is moved within the body using a dead reckoning process, which usually consists of a combination of sensors including an accelerometer to measure the object's acceleration and a gyroscope to measure the object's angular velocity. These sensors are often packaged in a single unit, called an inertial measurement unit (IMU). With the two sensors, the change of heading direction can be obtained by integrating the angular velocity, while the linear displacement can be obtained by integrating acceleration. However, the integration algorithm is subject to cumulative measurement error. To correct the accumulating error, the dead reckoning system is generally implemented with a Kalman Filter. This method requires indirect measurements to correct the target measurement. For example, an IMU based tracking method with an optical camera as the correction method. This method, however, is not feasible for positioning an object within the body. Furthermore, the target object will be moved slowly and smoothly during surgeries, which results in subtle acceleration and angular velocity that is close to the random noise level, which may cause cumulative inaccuracies.

A third method with high feasibility utilizes electromagnetic field (EMF). Compared with other waveforms, EMF can penetrate non-metal substances with little distortion and attenuation. More importantly, EMF has been shown to cause no harm to the human body when the strength is lower than 1 T. Currently, EMF is used for surgical tracking in two approaches. The first approach applies a passive coil as the sensor, then measures the current inducted by alternating EMF in an external reference electromagnet. The induced current amplitude at the sensor coil is related to its distance from the EMF source. Usually, several EMF sources are synthesized in a fixed pattern. The target location of the sensor can be obtained by processing the current reading with infinite impulse response filter.

The present approach is based on the magnitude measurement of a static EMF. The magnitude can be expressed as a three-component vector. The first step for locating the sensor is to measure magnetic field over an even distribution of measurement points in the target area. At each point, the EMF magnitude is measured and recorded along with its known location coordinate. New measurements from unknown locations are then compared with the recorded EMF pattern to find their closest matches, thereby the related locations can be determined. Early application of this idea was based on the geomagnetic field (GMF) in a large scale. For example, one example introduces the location sensing method inside a building. Their magnetic pattern is the local GMF, which is distorted by various structures in the building that makes the pattern unique among most locations. The drawback of utilizing the GMF is that it takes a great deal of labor to collect enough data for the EMF pattern. Also, the system is not reliable when the pattern varies with time, temperature, and weather. Finally, it is not feasible to acquire a priori magnetic field measurements in a human body application, particularly in a trauma situation.

In this example, the EMF approach is used with improvements over the drawbacks described above, especially as related to the surgical requirements. In the EMF method, a magnetic beacon is placed externally over a body landmark, for example, the xiphoid bone. This landmark is the destination for the sensors moving inside the torso. The receiver system inside the body consists of several EMF magnitude sensors. When the difference between all sensors' readings reaches the minimum, the sensors are right under the magnet beacon. This suggests the sensors inside have reached the destination. The drawback of this system is that it only gives a binary result of whether the sensor has reached the destination or not. There is, therefore, a need for an improved EMF tracking method with a controllable electromagnet as the source, along with special designs to guarantee its measurement accuracy and reliability.

### Location Measuring Procedures

Locating Principle with EMF

There are two stages in the method presented here using an EMF sensor. The first stage is to obtain the theoretical EMF reference around a certain magnet. The center of the magnet is treated as the origin of an orthogonal coordinate system, which can be called the magnet frame. The location of the detecting sensor can be expressed as a set of coordinates in this magnet frame. Every location coordinate is related with one EMF vector that was obtained theoretically. Next, in the measurement stage, new EMF measurements will be compared to the theoretical EMF pattern to find the closest match, which will be taken to be the measured location of the sensor.

The schematic of this method in surgical applications is shown in FIG. 18, where a body is shown as it would be in practice, lying on a flat surface. A body coordinate system 620 is shown with the first axis 622 (the X-axis) aligned with the aorta (along the length of the torso). A second axis 624 (the Y-axis) is transverse to the aorta and in the horizontal plane, and the third axis 626 (the Z-axis) is vertical (aligned with gravity). The origin of the body coordinate system 620 system is assumed to be aligned the centerline of the aorta, directly under the xiphoid, though in general it could be located at any point relative to any bony landmark. The goal in this application is to locate a specific portion of the perfusion stent (e.g. the leading edge) in the body coordinate system. FIG. 18 also shows an electromagnet located outside the body with a magnet coordinate system, with its axes 606, 608, 610 (X, Y, and Z axes, respectively). The algorithm, as described later, will determine the sensor location within the range of the electromagnet, expressed in the magnet coordinate system. If the magnetic coordinate system 600 is aligned with and at a known location relative to the body coordinate system 620, then one can transfer a known location in the magnet coordinate system 620 to the body coordinate system 620. This relative alignment and positioning is done by first placing the magnet 604 on or near the body in a known position and orientation relative to an appropriate landmark. In this example, the placement of the magnet is at the origin of the body frame, which is right above the xiphoid bone. For a cylinder magnet, the radial component of the EMF is isotropic. Therefore, by placing the magnet such that the Z axis is aligned vertically in space (aligned with the third axis 626 of the body coordinate system 620) the X axis in the magnetic coordinate system 600 (first magnetic axis 606) is naturally aligned to be parallel with the first axis 622 in body coordinate system 620 (whatever radial component that aligns with X_{b} is taken as the X axis). The direction of Y axis is thereby fixed. In this study, the destination of the sensor is directly under the xiphoid bone, in the aorta, though it could be set at any other location relative to the electromagnet within the measurable field.

The procedure for placement of the perfusion stent and accompanying sensor first involves inserting it into the body through an incision on the iliac artery and then pushing it cranially in the torso by a guide wire. After the stent enters the aorta, the trajectory can be approximated as a straight line. The goal of the locating system is to track the position of the sensor (and therefore the stent) along the aorta and report its position in the body frame. The detector is made from a printed circuit board (PCB) with all sensors soldered on. The schematic and the fabricated sensor is shown in FIGS. 19A-19B, and described in greater detail herein. In one example, the detector has a length of 30 mm and diameter of 1.8 mm. The stent will be also attached on the guide wire, with its relative position to the detector recorded. The implantable parts are contained inside a sheath, which in this example has diameter of 10 Fr (3.4 mm). When the sensor reaches the target location in vivo, the sheath will be removed, so that the stent will expand to cover the trauma on the vessel.

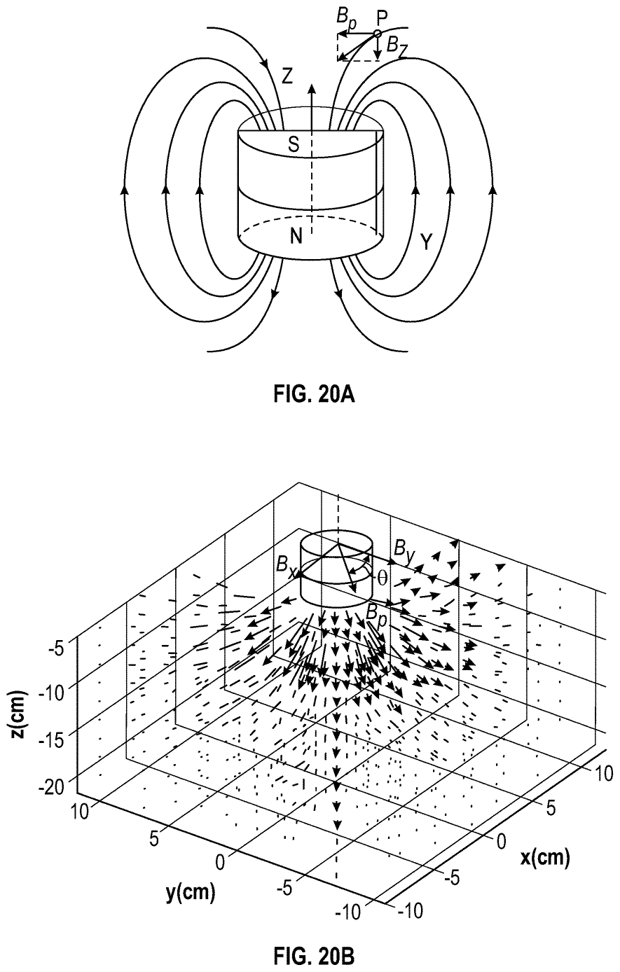

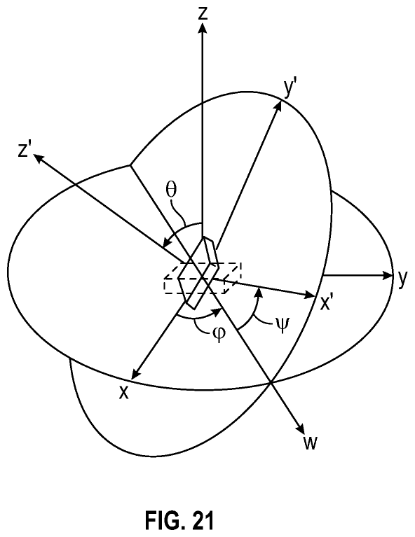

The reference magnetic field source is provided by a single cylindrical magnet 604, the most common shape for both permanent magnet and electromagnets, although multiple magnets and magnets of other shapes may be used. If the magnet 604 does not have a significant size, the cylinder can be simplified as two parallel planar coils, with current flowing in opposite directions (as shown in FIG. 20A). The simplified model allows an analytical expression for the magnetic field. For a cylindrical magnet 604 with height L and radius a, for any plane that passes through its central axis the magnetic field can be decomposed into an axial part B_{z} and a radial part Bₚ, which can be obtained by equations (1) and (2). Then by rotating Bₚ with angle θ around the Z axis, Bₚ is decomposed into Bₓ and B_{y} through equation (3) and (4). While B_{z} is unchanged with θ, the magnetic field is extended into the 3D coordinate system as shown in FIGS. 20A and 20B.

In equation (1) and (2), μ₀ is the constant of air permeability. The magnetic permeability of all human body components can be approximated by the permeability of water, whose difference to air permeability is negligible. Therefore, it is valid to treat the permeability as a static parameter. M is a characteristic parameter of the source magnet called magnetization that describes the magnetic moment in the unit volume.

$${B}_{z} = \frac{{μ}_{0}}{4 π} M {\int }_{0}^{2 π} {\int }_{0}^{a} ( \frac{R ( L / 2 - z )}{\begin{matrix}( {R}^{2} + {( L / 2 - z )}^{2} + {ρ}^{2} - \\ {2 R ρcosΦ )}^{3 / 2}\end{matrix}} + \frac{R ( L / 2 + z )}{\begin{matrix}( {R}^{2} + {( L / 2 + z )}^{2} + {ρ}^{2} - \\ {2 R ρcosΦ )}^{3 / 2}\end{matrix}} ) dRd Φ \qquad ( 1 )$$

$${B}_{ρ} = \frac{{μ}_{0}}{4 π} M {\int }_{0}^{2 π} {\int }_{0}^{a} ( \frac{R ( ρ - R cosΦ )}{\begin{matrix}( {R}^{2} + {( L / 2 - z )}^{2} + {ρ}^{2} - \\ {2 R ρcosΦ )}^{3 / 2}\end{matrix}} + \frac{R ( ρ - R cosΦ )}{\begin{matrix}( {R}^{2} + {( L / 2 + z )}^{2} + {ρ}^{2} - \\ {2 R ρcosΦ )}^{3 / 2}\end{matrix}} ) dRd Φ \qquad ( 2 )$$

$${B}_{x} = {B}_{ρ} cosθ \qquad ( 3 )$$

$${B}_{x} = {B}_{ρ} sinθ \qquad ( 4 )$$

For a permanent magnet, M can be treated as a constant; while for an electromagnet, Mis proportional to the instantaneous current amplitude. Since M is hard to measure directly, the product μ₀ M is simplified as constant C, which can be calibrated for the specific magnet of interest. To find its true value for a certain magnet, measurements are collected along a straight trajectory that passes the center of the magnet, then the ratio between the measurements and the analytical model at corresponding locations are calculated and then averaged, which is the value of C for this magnet.

Once the predicted magnetic field pattern is known, the next step is to locate new measurements in this pattern. In this example a nearest neighbor algorithm is used. Assume there are n reference points distributed in the magnetic field pattern. For the ith point, the location coordinate (Xᵢ, Yᵢ, Zᵢ) is related to the local magnetic field prediction (Bₓᵢ, B_{yi}, B_{zi}) using Equations 1-4. The magnetic field will be measured with a three-axis magnetometer, although multiple magnetometers may be used. When a measurement (B_{MX}, B_{MY}, B_{MZ}) is taken from the sensor at an unknown location, the difference Di between the measurement and reference values for location i=1, 2, 3, . . . , n is calculated by equation (5). Then an exhaustive search algorithm locates the sensor by determining the reference location where the minimum of Dᵢ occurs.

$${D}_{i} = \sqrt{{( {B}_{MX} - {B}_{Xi} )}^{2} + {( {B}_{MY} - {B}_{Yi} )}^{2} + {( {B}_{MZ} - {B}_{Zi} )}^{2}} \qquad ( 5 )$$

In the field application in which the magnet is placed right over the body landmark with axes aligned with the patient's body in the previously introduced way, the sensor's horizontal distance to the destination along the aorta is represented by the X axis coordinate. The measurement in this dimension is the most valuable for the stent placement application, however any preferred dimension may be used. The Y and Z axes readings, which represent the side and vertical location offsets from the magnet center, will not change drastically as the stent moves along the aorta.

### External Magnetic Source Cancellation

The working principles disclosed herein have been introduced under ideal conditions without noise and interference. In practice, various sources of magnetic interference are possible causes of error for all magnetic-based locating systems. However, the magnetic location method previously described can be adjusted to account for various sources of magnetic interference.

The first type of error is caused by near-sensor factors, which, in applications such as those disclosed herein, includes sensor manufacturing variance, interference of the printed circuit board (PCB) onto which it is soldered, and interference from the implantable device, such as the frame wires of the perfusion device 10 disclosed herein. These accuracy degrading factors can be classified as either soft-iron effects or hard-iron effects and can be expressed with the equation (6) below, with W representing the soft iron effects, such as soft iron materials in the environment around the sensors, and \vec{V} representing the hard-iron effects, such as interference from permanently magnetized objects positioned in the environment around the magnetometer.

$${\vec{B}}_{m} = W {\vec{B}}_{r} + \vec{V} \qquad ( 6 )$$

In this equation \vec{Bₘ} is a 3×1 vector of the three-axis sensor's actual measurement under interference, \vec{Bᵣ} is a 3×1 vector of the true magnetic field without interference at the same location, and W and \vec{V} are 3×3 and 3×1 matrices that contain constants that describe the soft and hard iron effects, respectively. Magnetic field distortion caused by ferromagnetic components that can be easily magnetized and de-magnetized by an external magnetic field is denoted as the soft-iron effect. In the application disclosed herein, the soft iron could come from the components on the PCB, or shrapnel inside the patient's body. Generally, it is assumed that the induced soft-iron field is linearly related to the source magnetic field. Therefore, the soft-iron effect can be represented by a 3×3 matrix W. There are still many factors whose effects can be represented by multiplying W with another 3×3 matrix. For example, the coordinate non-orthogonal error, and the different direction sensitivity error. Because of the complexity in both analyzing and calculating the components of W, it is assumed to be an identity matrix in most practical applications. For the condition where W cannot be simplified as an identity matrix, a comprehensive calibration method can be followed.

The hard-iron effect of the 3×1 vector \vec{V}=[V₁, V₂, V₃] represents the effect from all permanently magnetized components fixed around the magnetometer. Besides the components soldered on the PCB, interference could also come from the stent and the guide wire, which may be made from magnetic material. In addition, the magnetometer may have manufacturing bias, which can be combined with external interferences to make up the total constant bias \vec{V}. An example algorithm for solving \vec{V} requires the sensor to be held at a fixed location and simply rotated randomly to multiple k directions. The solution of \vec{V} can be obtained when the overall difference between all k measurements and the static magnitude reaches the minimum.

However, this method is not feasible for the methods disclosed herein, because the detector cannot be manipulated to rotate while working in the body. Therefore, an improved simulation of the interference equation is shown in the equation (7) below.

$${\vec{B}}_{m} = WI \vec{M} + \vec{V} + {\vec{V}}_{per} + I {\vec{V}}_{soft} \qquad ( 7 )$$

In this equation, the interference sources are classified into two categories, the first source is from the near-sensor range, which includes sensor manufacturing variance, the materials on the printed circuit board (PCB) onto which it is soldered, and the stent wires. After the detector is fabricated, the effect from these components will be constant, which are expressed by W and \vec{V}. The second source includes the interference in the background, the additional term \vec{Vₚₑᵣ} corresponds to the permanent disturbance that is invariant with time but variant with location. To remove the interference from \vec{Vₚₑᵣ}, an electromagnet is applied as the reference magnetic source. The amplitude of the field generated by an electromagnet is proportional to the running current amplitude I. Accordingly, \vec{Bᵣ} can be expressed by I\vec{M}, where \vec{M} is a constant vector based only on the properties of the electromagnet.

In the applications and methods disclosed herein, an electromagnet was chosen as the desired source because the magnetic field of an electromagnet can be adjusted by controlling the applied current. By toggling a relay (such as the relay 816 shown in FIG. 22) in the electromagnet circuit between the open and closed configuration, the current will switch between a constant amplitude I and 0, allowing for correction of the hard magnetic interference terms. At a given measurement location, when the switch is turned off, the background magnetic field is measured and denoted as \vec{M}₁=\vec{V}+\vec{V}ₚₑᵣ, then the current is turned off, and the new measurement

is denoted as \vec{M₂}, which includes background vector \vec{M₁} superposed on the desired magnetic reference vector. Therefore, the desired measurement, which is only contributed by the active magnet source and is denoted as \vec{M₀}, can be calculated as \vec{M₀}=\vec{M₂}−\vec{M₁}.

This switching process is repeated frequently while the detector is moving so that the process can compensate for an interference that varies with sensor location. Thus, the effectiveness of the method requires that \vec{M₁} and \vec{M₂} are measured at substantially the same location, which means the current switching should be frequent enough so that the displacement between two measurements in a dynamic process is negligible as the detector moves.

If an accelerometer is incorporated in the system as described above, its signal may be used to detect stationary moments during which the electromagnet may be made inactive and measurements may be made, or alternatively one may use sensor fusion (e.g., dead reckoning) as described above to know and account for the position change from state 1 to state 2. In some cases, the magnetic circuit time constant causes a delay of the magnetic field state from the current being switched off and on. This may be accounted for by waiting for sufficient time for the field to fully decay (when turned off) or reach steady-state (when turned on). Alternatively, one may incorporate measurement or estimate of the transient field state at any time (thereby increasing the frequency of location samples) through measurement of applied current to the electromagnet or through the use of a reference magnetometer inside or outside the body at a fixed and known location.

The soft-iron effect term W can be solved by calibration in advance or approximated as an identity matrix. The term I\vec{V}_{soft} is added in the above equation to represent soft-iron objects that are not fabricated with and located around the sensor. It is assumed that these interferences are caused by objects, for example a bullet or shrapnel, that are magnetized by the source magnet. Therefore, I\vec{V}_{soft} is a highly random term that depends on the number and magnetic susceptibility of all objects in the vicinity of the detector. Their effect will appear and disappear as the electromagnet source is switched on and off, and the amplitudes are proportional to the current amplitude running in the electromagnet. Assuming W is an identity matrix, and that the hard-iron interference has been successfully corrected for by the approach disclosed herein, the measurement error can be expressed by equation (8). At a stationary location, by adjusting and measuring the current amplitude I and then correspondingly varying \vec{B}ₘ, the slope of the I−|\vec{B}ₘ| curve is the magnitude of (\vec{M}+\vec{V}_{soft}). This approach cannot directly cancel the soft-iron object disturbance, but it can provide an indication of their magnitude and effect.

$${\vec{B}}_{m} = I ( \vec{M} + {\vec{V}}_{soft} ) \qquad ( 8 )$$

### Tilt Angle Compensation

In the process of deploying the sensor inside the artery, the sensor's linear translation may be accompanied with rotation. To obtain the closest matching point with a given measurement, the sensor's local coordinate frame is preferably kept aligned with the magnetic coordinate system 600 (this assumes a straight-line path of the aorta in the present application). As is shown in FIG. 21, for an arbitrary orientation of the sensor in the body, the fixed magnet frame (X, Y, Z) can be rotated to the direction of the sensor frame (X′, Y′, Z′) by first rotating around the X axis with angle φ, then rotating around Y axis with angle θ, and finally rotating around Z axis with angle ψ. These three angles are Euler angles and are usually called roll, pitch, and yaw. In this application, the stent is inserted through a non-solid guiding wire to move inside the thin and straight artery. These characteristics of the patient's anatomy make the detector rotation most likely to happen around the X axis, the roll angle. Therefore, the roll angle measurement is the most important secondary measurement in this system.

To measure these Euler angles, additional sensors can be added to the detector. Two types of sensors may be suitable for the measurement of Euler angles. One such method is to include a gyroscope that measures the angular velocity. The angular velocity can be continuously integrated to obtain the change in the Euler angles. However, this method can introduce cumulative measurement errors resulting from drift in the gyroscope.

Alternatively, an algorithm that uses an accelerometer may be applied. Before the sensor is used in practice, calibration may be necessary for an accelerometer. The accelerometer measurement error can be simplified as a constant bias in the way expressed by (9), where \vec{aₘ} is the measurement, \vec{aᵣ} is the real acceleration, and \vec{b} is the bias error.

$${\vec{a}}_{m} = {\vec{a}}_{r} + \vec{b} \qquad ( 9 )$$

To solve the X axis component of \vec{b}, which is denoted as bₓ, the X axis of the sensor can be first aligned vertically, with the X axis reading denoted as aₓ₁. Then the sensor can be flipped by upside down, with the X axis rotated for 180°, with the second reading denoted as aₓ₂. bₓ can be obtained as bₓ=(aₓ₂+aₓ₁)/2. The other two components can be obtained in the same way. The accelerometer signal contains both a static component (acceleration of gravity, 1 g) and a dynamic component (acceleration of the sensor as it is moved). While the sensor is approximately in its static state, or during very slow movements, the dynamic component is minimal and the measurement only consists of the gravity acceleration. However, the stent deployment process is a dynamic process, where the sensor is repeatedly moved, then stopped, then moved again. Therefore, in practice the movement state is determined as either static or dynamic by setting a threshold, for example a₀=g+/−0.05 g or a₀=g+/−0.1 g, as the static acceleration threshold. When the static condition is satisfied, the gravity acceleration G in the magnet frame and the measurement Gₚ in the sensor frame can be expressed by (10). This equation shows how to mathematically convert from one frame to another frame through a certain rotation sequence in roll, pitch, and yaw.

$${\overline{g}}_{p} = {R}_{X} ( φ ) {R}_{Y} ( θ ) {R}_{Z} ( ψ ) \overline{g} \qquad ( 10 )$$

$$\overline{g} = ( \begin{matrix}0 \\ 0 \\ g\end{matrix} ) \qquad ( 11 )$$

$${\overline{g}}_{p} = ( \begin{matrix}{a}_{x} \\ {a}_{y} \\ {a}_{z}\end{matrix} )$$

With the rotation matrix being:

$${R}_{X} ( φ ) = [ \begin{matrix}1 & 0 & 0 \\ 0 & cosφ & sinφ \\ 0 & sinφ & cosφ\end{matrix} ] \qquad ( 12 )$$

$${R}_{Y} ( θ ) = [ \begin{matrix}cosθ & 0 & sinθ \\ 0 & 1 & 0 \\ - sinθ & 0 & cosθ\end{matrix} ] \qquad ( 13 )$$

$${R}_{Y} ( ψ ) = [ \begin{matrix}cosψ & sinψ & 0 \\ - sinψ & cosψ & 0 \\ 0 & 0 & 1\end{matrix} ] \qquad ( 14 )$$

R_{Z}(ψ)\vec{G}=\vec{G}, which suggests the gravity acceleration measurement will not be affected by the change of ψ. In other words, w cannot be measured with gravity acceleration. Therefore, it is possible to first solve φ and θ by expanding the matrix to get the solution of (15) and (16). Note that for periods during which the measurement is greater than a₀, then the most recently found roll and pitch angles are used as approximations. As mentioned above, during stent deployment these periods are intermittent and not problematic.

$$φ = {tan}^{- 1} ( - \frac{{a}_{y}}{{a}_{z}} ) \qquad ( 15 )$$

$$θ = {tan}^{- 1} \frac{{a}_{x}}{\sqrt{{a}_{y}^{2} + {a}_{z}^{2}}} \qquad ( 16 )$$

To determine the yaw ψ, the help of geomagnetic field is used, where 0° is defined as the direction of the geomagnetic north pole. Since the magnetometer and accelerometer are fixed on the PCB to keep the three axes parallel to each other, the rotation matrix of (12) and (13) can be applied to rotate a random direction measurement of the geomagnetic field vector Bₘ=[Bₘₓ, B_{my}, B_{mz}] into a horizontal plane, where the vector \vec{Bₚ} has a Z axis component of 0. From the X and Y components of \vec{Bₚ}, the yaw angle can be obtained, the equation (17) for which is shown below:

$$ψ = {tan}^{- 1} ( - \frac{{B}_{py}}{{B}_{px}} ) = {tan}^{- 1} ( \frac{{B}_{mz} sinφ - {B}_{my} cosφ}{{B}_{mx} cosθ + {B}_{my} sinθsinφ + {B}_{mz} sinθcosφ} ) \qquad ( 17 )$$

Finally, when the Euler angles are solved, for a measurement \vec{Bₘ} that is heading to a random direction, the tilt compensation can be applied with the equation (18):

$$\vec{B} = {R}_{x}^{- 1} ( φ ) {R}_{y}^{- 1} ( θ ) {R}_{z}^{- 1} ( ψ ) {\vec{B}}_{m} \qquad ( 18 )$$

### Experimentation

### Ideal Test Condition

In vitro experiments were conducted to validate the system's performance. All parts used in the system are listed in Table I. The entire system is controlled by a Raspberry Pi, whose tasks include data collection, EMF control, location searching and display of results. The suitably small magnetometer and accelerometer are wired to the Raspberry Pi through an I2C bus.

TABLE 1
Electrical Parts Used in the System.

| Part Name | Manufacturer | Model Number |
|---|---|---|
| Controller | Raspberry Pi | Raspberry Pi 4 Model B |
| Magnetometer | MEMSIC | MMC5603NJ |
| Accelerometer | MEMSIC | MXC400xXC |
| Current sensor | Adafruit | INA219 |
| Digital relay | P&B Brand | RTE24005F |
| Electromagnet | BUNTING | BDE-3020-12 |
| DC power supply | MORNSUN | SE-350-24 |

The electromagnet is powered by an external power supply, and connected to the Raspberry Pi through a relay, which can be switched on and off to control the current flow. In the experiment, the current switching frequency is 5 Hz. An electromagnet is expected to have dynamics that create a delay between the input power application and the resulting field response. The electromagnet used in these tests has a response time of 0.04 to 0.08 seconds, which limits the frequency at which it can be switched. The cylinder magnet has diameter of 7.5 cm and height of 5 cm, and a maximum working DC voltage of 24V. The magnetic field pattern is created in a 40 cm×40 cm×20 cm space, then discretely sampled with 0.2 cm resolution in each dimension to create the reference magnetic field, similar with the pattern shown in FIGS. 20A and 20B. This range is large enough to cover the operational area of interest on a patient's body (that is, the portion of the patient's torso adjacent to the xiphoid bone in the example methods disclosed herein). Additionally, the magnitude of the generated field within this operational range is at least 20 times greater than the magnitude of the standard deviation of magnetometer measurement noise, further improving the reliability of the measurements.

The temperature of the electromagnet 802 will increase with the working time (that is, the time that current is supplied to the electromagnet 802). This will result in an increase in coil resistance and a corresponding decrease in current amplitude, which will proportionally change the magnitude of the magnetic field. Therefore, a current sensor can also be included, for example as a component of the controller 810, to monitor the real-time current amplitude.

A schematic view of the test system is presented in FIG. 22, and described in greater detail herein. FIG. 23 illustrates the test system as constructed for this example. As introduced previously, the stepper motor 812 is used to drive the detector 804 to known locations. At these known locations, the detector 804 will measure the magnetic field generated by the electromagnet 802, values for which are known. These measurements of the magnetic field will be to check the accuracy of the measurement at known control positions. The motor 812 will drive the detector 804 to move in a single axis on the rail 808 as described herein. The rail 808, as shown in FIG. 23, simulates the aorta of the patient, whose direction is aligned in parallel with the X axis in the magnet frame, however it is to be understood that, in principle, any pathway through the vasculature of the patient could be simulated in a similar fashion. In this simplified condition, the coordinate change along the rail 808 only occurs in the X axis component, with the other two components being static. For the last step, the detector will be deployed into a water-filled plastic tube and covered by animal flesh to simulate the in-vivo condition.

The first experiment involves several tests in which the magnet is fixed at different heights above the sensor. The testing range on the rail starts from 15 cm to the left of the magnet (at location −15 cm) and ends at 15 cm to its right (at location +15 cm). The experiment runs by positioning the stepper motor so that the sensor is at location-15 cm, then moving it to +15 cm in 0.2 cm increments, pausing at each increment (at which point a measurement is made). In the magnet frame, this can be expressed by the X component of its coordinate change from −15.0 to +15.0. The X component of the coordinate change represents the location of the sensor inside the artery, and so is of particular interest to this application. At each pause, the position detection algorithm is executed. Namely, the current to the electromagnet is turned off to update the background magnetic field and the search algorithm is run on the Raspberry Pi to calculate the sensor's position.

The measurement results along the three axes are shown for different heights of the electromagnet in Table 2. For Table 2 and in the rest of this section, the measurement performance will be evaluated by three factors, which are the overall averaged error, its standard deviation, and the error when the sensor is under the center of the magnet. These three factors will be put in a parenthesis as it shown in Table 2. In this example, the three evaluation errors in the X direction (0.16, 0.083, 0.12), is the smallest among the three directions. The results fall within the nominal requirements for placing a stent. In comparison, there is a larger error along Y at (0.72, 0.70, 0.57), and Z axis at (0.19, 0.32, 0.2), especially near the beginning of the test (at the left side of the operating range). Plots of the measurement data collected in this example are presented in FIGS. 24A through 24C, showing the measurement along the x-axis (FIG. 24A), the measurement along the y-axis (FIG. 24B), and the z-axis (FIG. 24C) as the sensor traverses in the x-axis direction. As shown in FIGS. 24A through 24C, there is close correlation between the reference position and the measured position in the absence of external interference.

TABLE 2
Measurement Error (CM) Evaluation at Different Heights.

| Height | X axis | Y axis | Z axis |
|---|---|---|---|
| 18 | (0.16, 0.083, 0.12) | (0.72, 0.70, 0.57) | (0.19, 0.32, 0.2) |
| 21 | (0.15, 0.11, 0.15) | (0.85, 0.67, 0.18) | (0.15, 0.12, 0) |
| 25 | (0.36, 0.31, 0.01) | (1.10, 0.71, 1.2) | (0.57, 0.79, 0.6) |

### Validation of Magnetic Field Interference Cancelling

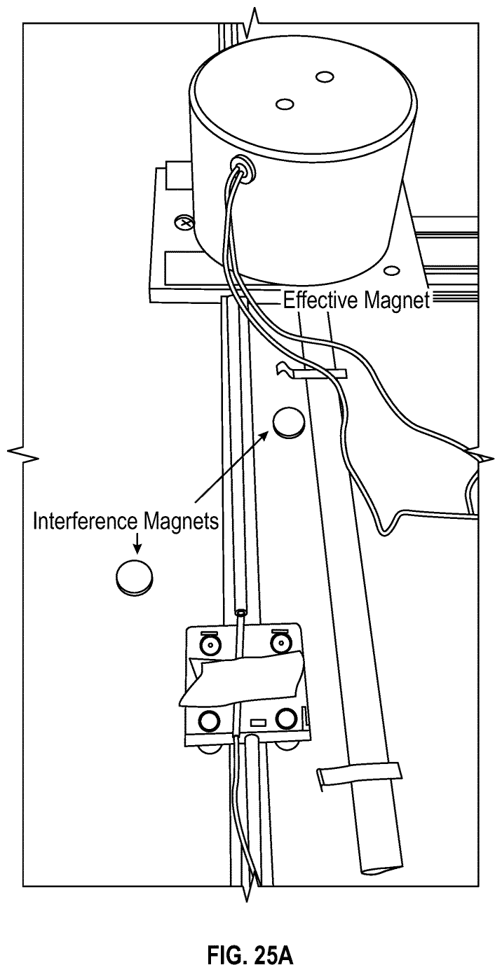

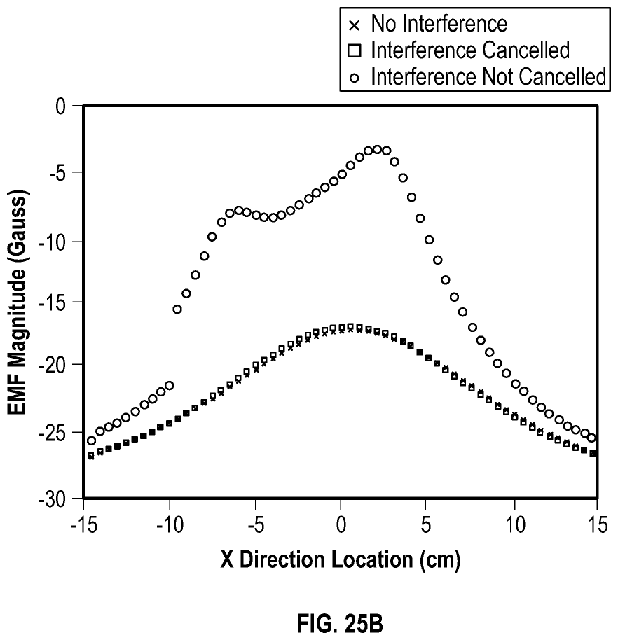

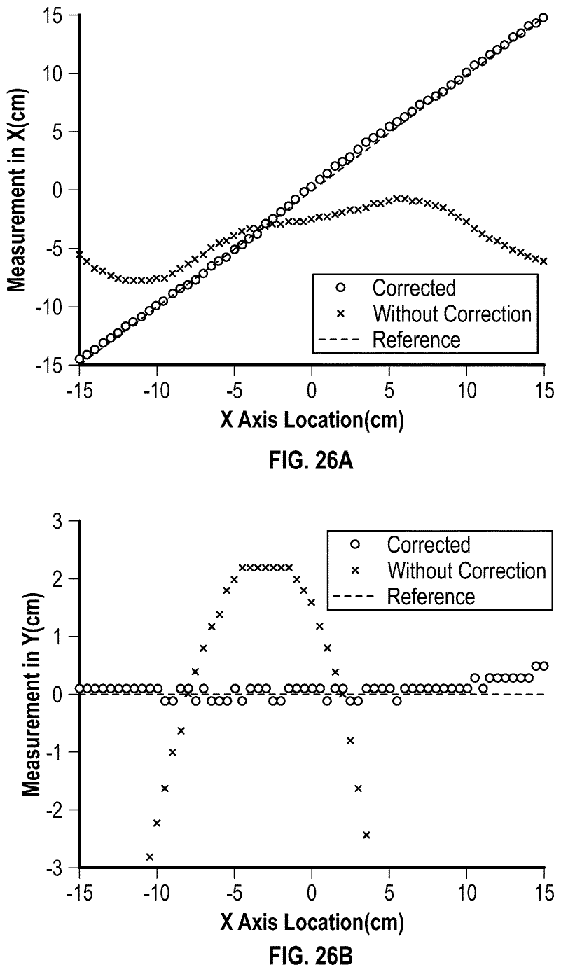

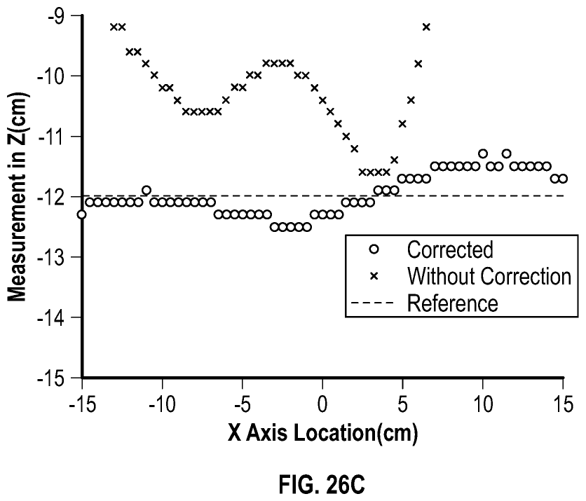

Based on the ideal condition test, extra magnetic interference is then introduced by placing two permanent magnets at random locations along the moving trajectory, as shown in FIG. 25A. To validate the effectiveness of the current-switching method in cancelling background interference, the magnetic field measurement with interference magnets is compared with data from a test in which they are not present. From the comparison shown in FIG. 25B, with the interference cancelling method, the maximum deviation is reduced from 3.25 to 0.033 Gauss, which suggests that the method successfully removes the external distortion and leaves the desired magnetic field to be used for location calculation. The location measurements, both with and without the interference correction factor, compared to the reference position, are presented in FIGS. 26A through 26C, showing the measurement along the x-axis (FIG. 26A), the measurement along the y-axis (FIG. 26B), and the z-axis (FIG. 26C) as the sensor traverses in the x-axis direction.

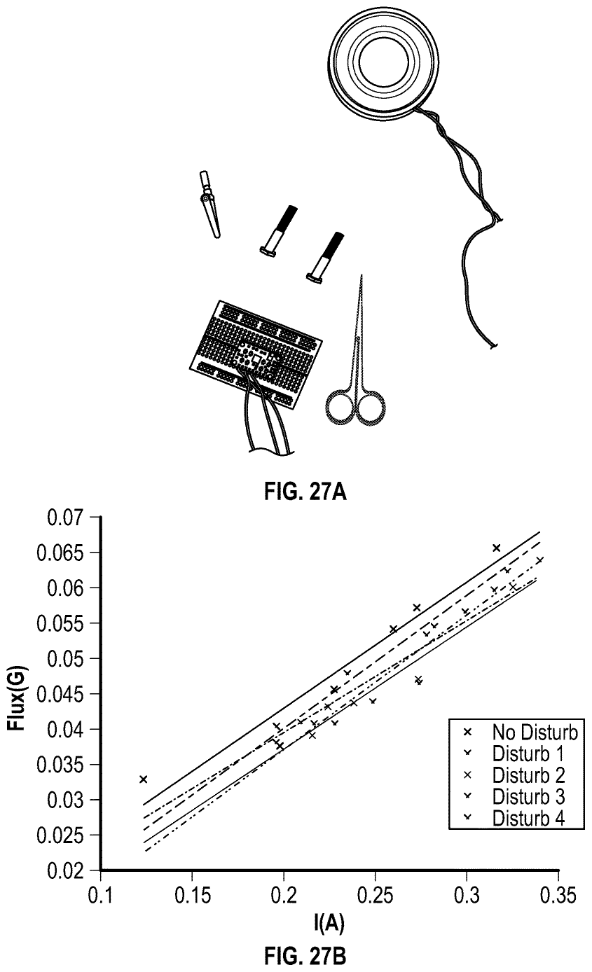

Turning now to FIG. 27A, a test setup to measure the amplitude of soft-iron interference from the objects around the sensor is shown, with the results shown in FIG. 27B. The soft-iron interference is introduced by four ferrous objects placed one after another around the magnetometer in the order shown in the figure, the disturbance term in equation (8) is changed from \vec{V}_{soft1} to the composite term \vec{V}_{soft1}+\vec{V}_{soft2}+\vec{V}_{soft3}+\vec{V}_{soft4}. As introduced before, the magnitude of this term is the slope parameter in the linear relation between I and |\vec{B}ₘ|. The electromagnet and the sensor are kept stationary so that their relative location does not change in the whole process. After each disturbing object is added in, the current is randomly adjusted to five different values and measured by the current sensor. The magnitude of the field is measured correspondingly. A linear curve is determined based on the five measurements using the curve fitting approach. In the result shown in FIG. 10(b), the slopes of the five experiments are calculated as 0.178, 0.189, 0.173, 0.159 and 0.188. Compared to the first experiment whose slope is 0.178, the largest difference occurs in the fourth experiment whose slope is 0.159. The ratio between these two experiments is 0.893, which means at the worst condition, the field measurement is 0.893 times of the actual field due to the soft-iron disturbances. When the magnetic measurement data from the ideal condition test introduced in FIG. 7) is multiplied by the ratio of 0.893, the average location measurement variant in the three directions are (0.29, 0.019, 0.69) cm. In addition, the location variants in the three directions are smallest, (−0.02, 0.019, 0.63) cm, at the target location, and largest, (0.6, 0.2, 0.8) cm, at the initial location. The test shows the soft-iron effect from common ferrous objects is not a significant cause of error in this application.

### Validation of the Tilt Angle Compensation

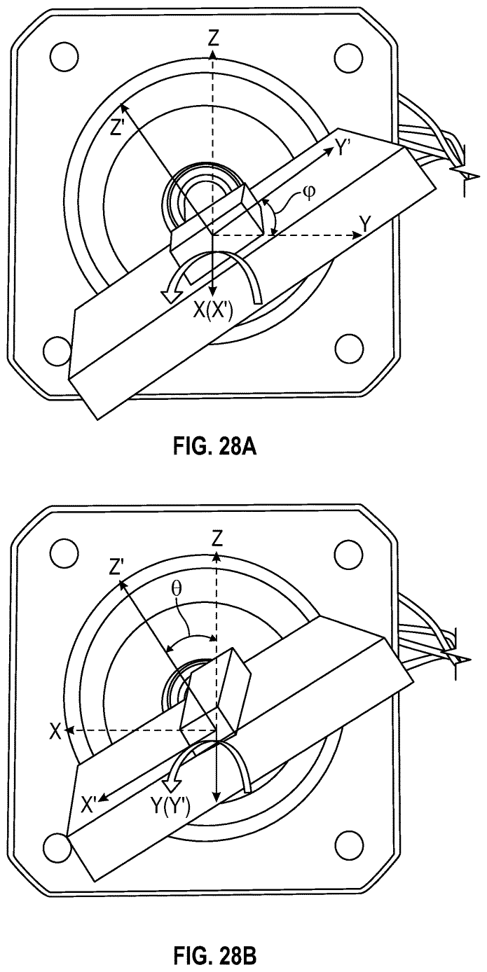

If the roll, pitch, yaw angle can be measured accurately during a rotation, the tilt compensation algorithm can be validated. An experiment was run in which a stepper motor was used to drive the sensor to known angles. These angles were compared with the calculated angle through equation (15), (16) and (17) to validate the function of accelerometer and magnetometer. The roll, pitch, yaw angles are validated separately by aligning each axis of the sensor in turn to the motor shaft as it shown in FIGS. 28A and 28B. For roll and pitch angle, 0° is defined when the Z axis is aligned vertically. The 0° for yaw angle is defined when the X axis is aligned with North pole of the GMF. Clockwise rotation is denoted as positive, and counter-clockwise rotation is denoted as negative. Based on practical experience, when the sensor moves along the thin and nearly straight artery, the pitch and yaw angle will be restricted to a small value, usually less than 15°. However, rotation around the X axis is less restricted. Therefore, the roll angle φ is validated in a larger range compared with pitch angle θ and yaw angle ψ.

The results are shown in Table 3 and Table 4, which show the effect of rotation on the average locating error with and without the angle compensation. The columns of the tables show the actual angles imposed during the test, the calculated angles based on accelerometer, the increased X-axis average error without the calculated tilt, and finally the X-axis error when the measured tilt is compensated. The inclusion of the angle compensation algorithm effectively reduces the locating error, particularly for the cases in which roll angles are introduced. As expected, the error becomes more drastic at large angles (e.g., over 4 cm for 135° of roll or 20° of pitch). In all cases, the compensation reduced the errors due to tilt angles to about 0.2 cm for roll and about 0.5 cm for pitch. For comparison, refer to the magnetic locating test shown in Table 2. When no rotation occurs, the average error in the X direction was between 0.16 and 0.36 cm across the 30 cm path.

TABLE 3
Roll (φ) measurement and its effect on locating.

| Motor setting | φ | Error without | Error with |
|---|---|---|---|
| reference | Measurement | correction | correction |
| (Degree) | (Degree) | (cm) | (cm) |
| −135 | −134.2 | 4.16 | 0.22 |
| −90 | −85.1 | 0.89 | 0.23 |
| −45 | −47.7 | 0.45 | 0.21 |
| 0 | 1.6 | 0.16 | 0.23 |
| 45 | 46.8 | 0.34 | 0.23 |
| 90 | 85.3 | 0.66 | 0.23 |
| 135 | 139.9 | 2.54 | 0.22 |

TABLE 4
Pitch (θ) measurement and its effect on locating.

| Motor setting | θ | Error without | Error with |
|---|---|---|---|
| reference | Measurement | correction | correction |
| (Degree) | (Degree) | (cm) | (cm) |
| −20 | −22 | 4.61 | 0.47 |
| −10 | −12.5 | 2.34 | 0.56 |
| 0 | −3 | 0.22 | 0.47 |
| 10 | 7 | 2.34 | 0.34 |
| 20 | 16.5 | 4.56 | 0.67 |

### All Interferences Synthesized

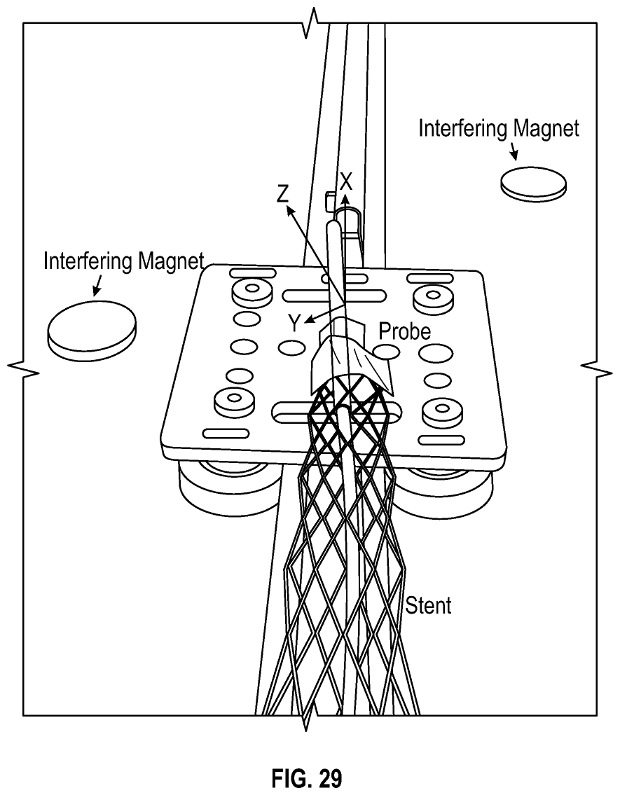

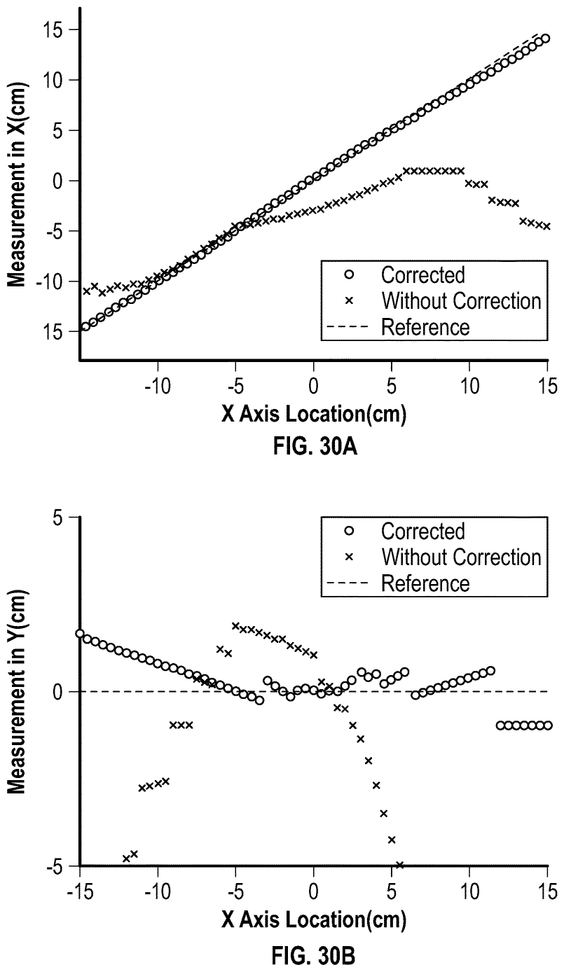

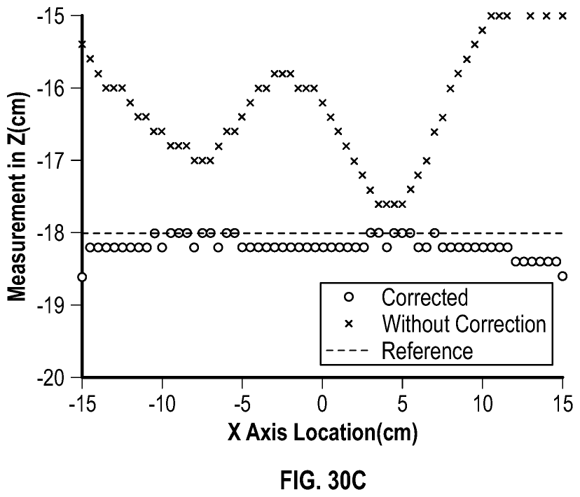

Finally, a test was designed in which all of the above-mentioned disturbances were included. The test setup is shown in FIG. 29, the magnet source was placed 18 cm vertically above the sensor. The sensor's Z axis was not aligned vertically. The detecting probe was attached to the stent. Two extra magnets were placed along the rail as a source of interference. The results are shown in FIGS. 30A through 30C for both the case with no compensation, and for the case in which the compensation methods described above are used. Compared with the measurement using raw data, as plotted in FIGS. 30A-30C, the correction methods can greatly reduce the deviation. When the correction methods are applied, the error along X, Y, Z directions, represented by the averaged error, standard deviation, and zero point error, are respectively (0.24, 0.22, 0.01), (0.50, 0.43, 0.01), (0.18, 0.13, 0.2). These results are well within the desirable range, particularly around the target location, with the error at the center point is just 0.01 cm.

### Discussion and Conclusion

The robustness of the system disclosed above was tested against various disturbances and non-idealizations. The overall average error, being less than 0.5 cm, is acceptable for the application of interest. More importantly, the measurement when the sensor locates vertically under the magnet is especially accurate (near ideal situation), which guarantees accuracy when it is used as the location landmark.

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A medical assembly, comprising a delivery apparatus comprising a sheath and a guidewire, configured to be advanced through vasculature within a body of a patient, a perfusion device having a radially expandable stent comprising a frame and a sealing cover disposed along the frame, a magnet, and a detector comprising a magnetometer; wherein the magnet is configured to be positioned external to the body of the patient and to generate a known magnetic field; wherein the stent and the detector are positioned on the guidewire and configured to be advanced along the guidewire and deployed from the sheath; wherein the magnetometer is configured to measure the known magnetic field and to identify a position of the detector relative to the magnet.

Example 2. The medical assembly any example herein, particularly example 1, wherein the magnet is an electromagnet and wherein the electromagnet may be switched between an active state to a non-active state.

Example 3. The medical assembly any example herein, particularly example 1, wherein the detector is positioned distal to or proximal to the stent on the guidewire.

Example 4. The medical assembly any example herein, particularly example 1, wherein the detector further comprises an accelerometer or a gyroscope.

Example 5. The medical assembly any example herein, particularly example 4, wherein the accelerometer or the gyroscope is configured to measure a position of the detector relative to a known reference position.

Example 6. The medical assembly any example herein, particularly example 4, wherein the accelerometer or the gyroscope is used to calculate at least one of a yaw, a pitch, or a roll of the detector.

Example 7. The medical assembly of any example herein, particularly example 4, wherein the accelerometer or the gyroscope are a triple-axis sensor.

Example 8. The medical assembly any example herein, particularly example 1, wherein the magnetometer is a triple-axis magnetometer.

Example 9. The medical assembly any example herein, particularly example 1, wherein the magnet is a first magnet, the known magnetic field is a first known magnetic field, and wherein the medical assembly comprises a second magnet configured to be positioned external to the body of the patient at a location spaced apart from the location of the first magnet, and to generate a second known magnetic field.

Example 10. The medical assembly any example herein, particularly example 1, further comprising a microcontroller that receives measurements from the magnetometer.

Example 11. The medical assembly any example herein, particularly example 10, wherein the microcontroller is configured to perform a correction operation on the measurements received from the magnetometer.

Example 12. A method for treating a vascular injury of a patient, comprising placing a magnet at a reference point on or near a body of the patient and generating a reference magnetic field and an origin point for the reference magnetic field; determining the location of a body landmark relative to the origin of the reference magnetic field; inserting a medical assembly comprising a perfusion stent and a detector into the patient's vasculature and advancing the medical assembly towards the location of the vascular injury; using the detector to measure the reference magnetic field and determine a position of the medical assembly relative to the origin point of the reference magnetic field; and deploying the perfusion stent once the medical assembly has reached the prescribed location in the body relative to the body landmark.

Example 13. The method any example herein, particularly example 12, further comprising adjusting the position of the medical assembly within the patient based on the position of the medical assembly relative to the origin point of the reference magnetic field.

Example 14. The method any example herein, particularly example 12, wherein the detector comprises a magnetometer, and wherein determining the position of the medical assembly relative to an origin of the reference magnetic field comprises comparing a first magnetic field measurement to a known reference magnetic field value for the reference magnetic field.

Example 15. The method any example herein, particularly example 14, wherein determining the position of the medical assembly relative to the origin of the reference magnetic field further includes compensating for one or more of a roll, a pitch, or a yaw of the medical assembly.

Example 16. The method any example herein, particularly example 12, further comprising using an accelerometer to measure changes in position of the medical assembly.

Example 17. The method any example herein, particularly example 12, further comprising closing the vascular injury and retracting the perfusion stent from the location of the vascular injury after the vascular injury has been closed.

Example 18. The method any example herein, particularly example 12, further comprising switching the magnet between an active state and a non-active state to generate an reference magnetic field when the magnet is in the active state to determine a background magnetic field when the magnet is in the non-active state, and using the reference magnetic field and the background magnetic field to calculate a correction factor based on a difference between the reference magnetic field and the background magnetic field.

Example 19. A magnetic location detection system, comprising a reference magnet; a detector including a magnetometer; and a microcontroller in communication with the detector; wherein the reference magnet is placed external to a patient and configured to generate a reference magnetic field with a known origin, a known magnitude, and a known direction; wherein the magnetometer is configured to measure a first magnitude and a first direction of the reference magnetic field at a first position, and wherein the microcontroller is configured to determine the first position of the magnetometer relative to an origin of the reference magnetic field using a measurement of the first magnitude and the first direction of the reference magnetic field.

Example 20. The magnetic location detection system any example herein, particularly example 19, wherein the detector further comprises an accelerometer, and wherein the microcontroller is configured to receive data from the accelerometer and to calculate one or more of a pitch or a roll of the detector.

Example 21. The magnetic location detection system any example herein, particularly example 19, wherein the reference magnet is an electromagnet which can be transitioned between an active state and an inactive state by applying a current to the electromagnet.

The features described herein with regard to any example can be combined with other features described in any one or more of the examples, unless otherwise stated. For example, any one or more of the features of one frame or actuator can be combined with any one or more features of another frame or actuator.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A medical assembly, comprising:
a delivery apparatus comprising a sheath and a guidewire, configured to be advanced through vasculature within a body of a patient;
a perfusion device having a radially expandable stent comprising a frame and a sealing cover disposed along the frame;
a magnet; and
a detector comprising a magnetometer;
wherein the magnet is configured to be positioned external to the body of the patient and to generate a known magnetic field;
wherein the radially expandable stent and the detector are positioned on the guidewire and configured to be advanced along the guidewire and deployed from the sheath;
wherein the magnetometer is configured to measure the known magnetic field and to identify a position of the detector relative to the magnet.

2. The medical assembly of claim 1, wherein the magnet is an electromagnet and wherein the electromagnet may be switched between an active state to a non-active state.

3. The medical assembly of claim 1, wherein the detector is positioned distal to or proximal to the radially expandable stent on the guidewire.

4. The medical assembly of claim 1, wherein the detector further comprises an accelerometer or a gyroscope.

5. The medical assembly of claim 4, wherein data from the accelerometer and or the gyroscope is used to determine the position of the detector relative to a known reference position.

6. The medical assembly of claim 4, wherein data from the accelerometer or the gyroscope is used to calculate at least one of a yaw, a pitch, or a roll of the detector.

7. The medical assembly of claim 1, wherein the magnetometer is a triple-axis magnetometer.

8. The medical assembly of claim 1, wherein the magnet is a first magnet, the known magnetic field is a first known magnetic field, and wherein the medical assembly comprises a second magnet configured to be positioned external to the body of the patient at a location spaced apart from the location of the first magnet, and to generate a second known magnetic field.

9. The medical assembly of claim 1, further comprising a microcontroller that receives measurements from the magnetometer.

10. The medical assembly of claim 9, wherein the microcontroller is configured to perform a correction operation on the measurements received from the magnetometer.

11. A method for treating a vascular injury of a patient, comprising:
placing a magnet at a reference point on or near a body of the patient and generating a reference magnetic field and an origin point for the reference magnetic field;
determining a location of a body landmark relative to the origin point of the reference magnetic field;
inserting a medical assembly comprising a perfusion stent and a detector into a vasculature of the patient and advancing the medical assembly through the vasculature of the patient towards the reference point on the body;
using the detector to measure the reference magnetic field and determine a position of the medical assembly relative to the origin point of the reference magnetic field; and
deploying the perfusion stent once the medical assembly has reached a prescribed location in the body relative to the body landmark.

12. The method of claim 11, further comprising adjusting the position of the medical assembly within the patient based on the position of the medical assembly relative to the origin point of the reference magnetic field.

13. The method of claim 11, wherein the detector comprises a magnetometer, and wherein determining the position of the medical assembly relative to an origin of the reference magnetic field comprises comparing a first magnetic field measurement to a known reference magnetic field value for the reference magnetic field.

14. The method of claim 13, wherein determining the position of the medical assembly relative to the origin of the reference magnetic field further includes compensating for one or more of a roll, a pitch, or a yaw of the medical assembly.

15. The method of claim 11, further comprising using an accelerometer to measure changes in the position of the medical assembly.

16. The method of claim 11, further comprising closing the vascular injury and retracting the perfusion stent from the vasculature of the patient after the vascular injury has been closed.

17. The method of claim 12, further comprising:
switching the magnet between an active state and a non-active state to generate the reference magnetic field when the magnet is in the active state and measuring a background magnetic field when the magnet is in the non-active state, and
using the reference magnetic field and the background magnetic field to calculate a correction factor based on a difference between the reference magnetic field and the background magnetic field.

18. A magnetic location detection system, comprising:
a reference magnet;
a detector including a magnetometer; and
a microcontroller in communication with the detector;
wherein the reference magnet is placed external to a patient and configured to generate a reference magnetic field with a known origin, a known magnitude, and a known direction;
wherein the magnetometer is configured to measure a first magnitude and a first direction of the reference magnetic field at a first position, and
wherein the microcontroller is configured to determine the first position of the magnetometer relative to an origin of the reference magnetic field using a measurement of the first magnitude and the first direction of the reference magnetic field.

19. The magnetic location detection system of claim 18, wherein the detector further comprises an accelerometer, and wherein the microcontroller is configured to receive data from the accelerometer and to calculate one or more of a pitch or a roll of the detector.

20. The magnetic location detection system of claim 18, wherein the reference magnet is an electromagnet which can be transitioned between an active state and an inactive state by applying a current to the electromagnet.
